(19) United States
(12) Patent Application Publication
(10) Pub. No.: US 2025/0134889 A1
(43) Pub. Date: May 1, 2025

(54) TARGETING IRE1 KINASE AND FMRP FOR PROPHYLAXIS, MANAGEMENT AND TREATMENT OF ATHEROSCLEROSIS

(71) Applicant: CEDARS-SINAI MEDICAL CENTER, Los Angeles, CA (US)

(72) Inventors: Ebru ERBAY, Sherman Oaks, CA (US); Zehra YILDIRIM, Beverly Hills, CA (US)

(73) Assignee: CEDARS-SINAI MEDICAL CENTER, Los Angeles, CA (US)

(21) Appl. No.: 18/834,683

(22) PCT Filed: Feb. 10, 2023
(86) PCT No.: PCT/US2023/062366

§ 371 Date: Jul. 31, 2024

Related U.S. Application Data
(60) Provisional application No. 63/309,331, filed on Feb. 11, 2022.

(51) Int. Cl. A61K 31/506; A61P 9/00; G01N 33/50

(52) U.S. Cl. CPC A61K 31/506; A61P 9/00; G01N 33/5055

(57) ABSTRACT

Fragile X Mental Retardation protein (FMRP) is an RNA-binding protein (RBP) that controls translation of select mRNAs. We discovered that endoplasmic reticulum (ER) stress induces phosphorylation of FMRP on a site that is known to enhance translation inhibition of FMRP-bound mRNAs. We show ER stress-induced activation of inositol requiring enzyme-1 (IRE1), an ER-resident stress-sensing kinase/endoribonuclease, leads to FMRP phosphorylation and to suppression of cholesterol efflux from macrophages and apoptotic cell clearance (efferocytosis) by macrophages. Conversely, FMRP-deficiency and pharmacological inhibition of IRE1 kinase activity enhances cholesterol efflux and efferocytosis, reducing atherosclerosis in mice. Our results provide IRE1 inhibition as a promising new therapeutic approach to delaying progression or treating atherosclerosis.

### CROSS-REFERENCE TO RELATED APPLICATIONS

This application includes a claim of priority under 35 U.S.C. § 119 (e) to U.S. provisional patent application No. 63/309,331, filed Feb. 11, 2022, the entirety of which is hereby incorporated by reference.

### STATEMENT REGARDING FEDERALLY SPONSORED RESEARCH OR DEVELOPMENT

This invention was made with Government support under grant no. HL152156 awarded by the National Institutes of Health. The Government has certain rights in the invention.

### REFERENCE TO SEQUENCE LISTING

This application contains a sequence listing submitted as an electronic xml file named, “Sequence_Listing_065472-000888WOPT” created on Feb. 7, 2023 and having a size of 54,622 bytes. The information contained in this electronic file is hereby incorporated by reference in its entirety.

### FIELD OF INVENTION

This invention relates to small molecule inhibitors targeting IRE1-FMRP signaling pathway for prophylaxis and treatment of atherosclerosis.

### BACKGROUND

Atherosclerosis is a chronic inflammatory vascular disease resulting from maladaptive inflammatory response to an imbalanced lipid metabolism. In atherosclerotic plaques, macrophages ingest lipoproteins and transform into lipid-laden foam cells. The foamy macrophages lose their ability to migrate away from the plaques, where they sustain a local state of sterile inflammation, which occurs in the absence of pathogens and is typically associated with the release of immune-recognizable cellular content from damaged or dying cells. The cholesterol-laden, foamy macrophages found in plaques play a pivotal role in perpetuating the sterile inflammation that is characteristic of atherosclerosis. Transcriptional control plays an important role in setting into motion this sterile inflammation.

Cholesterol efflux and efferocytosis (the engulfment and clearance of apoptotic cells (AC)) by macrophages, on the other hand, help to resolve inflammation and contribute to plaque stability as counterbalancing mechanisms that oppose plaque rupture. Post-transcriptional mechanisms that operate in atherosclerosis can contribute to resolution of inflammation and promote plaque regression, presenting a therapeutic opportunity. Cholesterol efflux by plaque macrophages is the first step in a multi-step process, referred to as “reverse cholesterol transport” (RCT), that reduces lipid accumulation in plaques. Macrophages efflux intracellular cholesterol using their plasma membrane cholesterol transporters (such as the ATP-binding cassette (ABC) transporters subfamily A member-1 (ABCA1) and subfamily G member-1 (ABCG1)), which in turn hand the exported cholesterol over to lipid-poor apolipoproteins, forming high-density lipoprotein (HDL) particles. The cholesterol efflux pathway is transcriptionally activated by the metabolic by-products of AC-derived cholesterol in the efferocytic macrophages. As such, efferocytosis and RCT synergize to reduce necrosis and resolve inflammation in plaques.

Ribonucleic acid RNA-binding proteins (RBP) alter cytokine and chemokine messenger RNA (mRNA) stability or translation to fine-tune or turn-off the inflammatory response. RBPs also post-transcriptionally regulate key proteins for cholesterol homeostasis and lipid metabolism in macrophages and liver. However, RBPs have not been closely investigated in atherosclerosis. As an example, fragile X mental retardation protein (FMRP) is an RBP that has been studied in neurons in association with the Fragile X Mental Retardation Syndrome (FXS). In FXS, a hypermethylated CGG repeat expansion in the 5′ untranslated region (5′ UTR) of the FMR1 mRNA results in its transcriptional silencing. A key serine phosphorylation (S500 in human; S499 in mouse) on FMRP triggers hierarchical phosphorylation of surrounding serines and threonines, while enhancing FMRP's translation-repressing activity on many synaptic function-linked mRNAs bound by it. However, the identity of the kinase(s) that phosphorylates FMRP is illusive, and the role of FMRP in the atherosclerotic process has not been investigated.

Therefore, it is an objective of the present invention to provide compositions and methods to prevent atherosclerosis by fine-tuning the homeostatic stress response that is pathologically activated by hyperlipidemia.

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

### SUMMARY OF THE INVENTION

The present disclosure provides a method for reducing the likelihood or inhibiting progression of atherosclerosis, or reducing the severity or treating atherosclerosis, in a subject in need thereof. The method includes administering to the subject a composition comprising a therapeutically effective amount of an inhibitor of inositol-requiring enzyme-1 (IRE1), an inhibitor of fragile X mental retardation protein (FMRP), or both.

According to some embodiments, the inhibitor of IRE1 is a small molecule that inhibits kinase activity of the IRE1, wherein in the presence of the IRE1 and the inhibitor of IRE1, phosphorylation of FMRP is reduced compared to that in the presence of the IRE1 without the inhibitor of IRE1. For example, the inhibitor of IRE1 comprises ORIN1001, MCK8866, AMG-18, G-9807, G-1749, STF-083010, or 4μ8C. For example, the inhibitor of IRE1 is a kinase-specific inhibitor of IRE1, comprising AMG-18.

According to some embodiments, the inhibitor of IRE1 inhibits level of IRE1 by suppressing expression of ERN1 gene, wherein the inhibitor is a small interfering RNA (siRNA), a microRNA (miRNA) or an antisense oligo, wherein the siRNA, the miRNA, or the antisense oligo targets the mRNA of ERN1 and suppresses expression of at least kinase domain of ERN1.

According to some embodiments, the inhibitor of FMRP inhibits level of FMRP by suppressing expression of FMRP gene, wherein the inhibitor is an siRNA, a miRNA, or an antisense oligo, wherein the siRNA, the miRNA, or the antisense oligo targets FMR1 and suppresses expression of FMR1.

According to some embodiments, the subject has obesity. According to some embodiments, the subject has hyperlipidemia or hypercholestorelimia.

According to some embodiments, the composition is administered once, twice, or three times per day to the subject until one or more improvements is observed, said improvements comprising reduced atherosclerotic lesion, reduced foam cell area in the atherosclerotic lesion, and/or reduced necrotic core area of the atherosclerotic lesion, in a blood vessel of the subject.

The present disclosure provides a method for enhancing macrophage's activity in engulfing apoptotic cells for clearance, exporting cholesterol to lipid-poor apolipoprotein, and/or internalizing low density lipoprotein (LDL), reducing the likelihood of foam cell formation from the macrophage, and/or reducing atherosclerotic lesion or necrotic core area of the atherosclerotic lesion in a blood vessel, said atherosclerotic lesion comprising the macrophage, the method comprising contacting the macrophage with an inhibitor of IRE1, an inhibitor of FMRP, or both; or transfecting or expressing a vector encoding the inhibitor of IRE1, the inhibitor of FMRP, or both in the macrophage.

According to some embodiments, the inhibitor of IRE1 is a small molecule that inhibits kinase activity of the IRE1, wherein in the presence of the IRE1 and the inhibitor of IRE1, phosphorylation of FMRP is reduced compared to that in the presence of the IRE1 without the inhibitor of IRE1. For example, the inhibitor of IRE1 comprises AMG-18, G-9807, G-1749, STF-083010, or 4μ8C. For example, the inhibitor of IRE1 is a kinase-specific inhibitor of IRE1, comprising AMG-18.

According to some embodiments, the inhibitor of IRE1 inhibits level of IRE1 by suppressing expression of ERN1 gene, wherein the inhibitor is a small interfering RNA (siRNA), a microRNA (miRNA) or an antisense oligo, wherein the siRNA, the miRNA, or the antisense oligo targets the mRNA of ERN1 and suppresses expression of at least kinase domain of ERN1.

According to some embodiments, the inhibitor of FMRP inhibits level of FMRP by suppressing expression of FMRP gene, wherein the inhibitor is an siRNA, a miRNA, or an antisense oligo, wherein the siRNA, the miRNA, or the antisense oligo targets FMR1 and suppresses expression of FMR1. For example, the inhibitor of FMRP is a phosphatase which reduces pFMRP/FMRP ratio in the macrophage

According to some embodiments, the subject has obesity. According to some embodiments, the subject has hyperlipidemia or hypercholestorelimia.

The present disclosure provides a method for screening for an atheroprotective agent that inhibits kinase activity of IRE1, inhibits binding between IRE1 and FMRP, or reduces phosphorylation of FMRP. The method includes contacting a molecule of interest with an atherosclerotic lesion cell type, said cell type expressing IRE1 and FMRP, and measuring an amount of phosphorylation of the FMRP in the cell type. A decrease in the amount of phosphorylation of the FMRP relative to an amount before the contact with the molecule of interest is indicative that the molecule of interest is an atheroprotective agent. The atherosclerotic lesion cell type comprises macrophage, endothelial cell, or vascular smooth muscle cell.

According to some embodiments, the method further includes stressing the atherosclerotic lesion cell type by contacting the atherosclerotic lesion cell type with a stressor, said stressor comprising a saturated fatty acid, an oxidized low density lipoprotein (oxLDL), thapsigargin, or tunicamycin. For example, the atherosclerotic lesion cell type is macrophage.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIGS. 1A-1K depict FMRP is a new inositol-requiring enzyme-1 (IRE1) kinase substrate. A representative blot is shown from at least n=3 independent experiments. Data are mean±SEM. Unpaired 1-test with Welch's correction or paired 1-test. * P≤0.05, ** P≤0.01, *** P≤0.001.
1A. STING analysis of published IRE1 interactome proteins in relation to FMRP (Acosta-Alvear et al., 2018).
1B. HEK293T cells were co-transfected with IRE1 and FMRP plasmids and stimulated with TG (600 nM) or TM (1 mg/ml) for 2 hours. Protein lysates were immunoprecipitated (IP) with anti-IRE1 or IgG (control) antibodies and analyzed by Western blotting using specific antibodies for FMRP and IRE1 (n=3).
1C. RAW 264.7 mouse macrophages were treated with either oxLDL (50 μg/ml) or TG (300 nM) for 6 hours. Protein lysates were treated with λ Phosphatase (PPase) for 30 minutes and analyzed by western blotting using specific antibodies for pFMRP, FMRP, pIRE1, IRE1 and β-Actin. pFMRP/FMRP fold induction is depicted above the blots (n=6).
1D. Apoe^{-/-} mice were fed with chow diet (CD) or western diet (WD) for 16 weeks followed by peritoneal macrophage (PM) isolation. Protein lysates were analyzed by western blotting using specific antibodies for pFMRP, FMRP, pIRE1, IRE1 and β-Actin. pFMRP/FMRP-fold induction is depicted above the blots (n=5).
1E. Control- or IRE1-siRNA transfected HEK293T cells were stimulated by either PA (500 μM) or TG (600 nM) for 4 hours. Protein lysates were analyzed by western blotting using specific antibodies for pFMRP, FMRP, pIRE1, IRE1 and β-Actin. pFMRP/FMRP fold induction is depicted above the blots (n=4).
1F. Protein lysates of thioglycolate-elicited PM from IRE1α^{-/-} and IRE1α^{-/-} mice (after 16 weeks on WD) were analyzed by western blotting using specific antibodies for pFMRP, FMRP, pIRE1, IRE1 and β-Actin. pFMRP/FMRP fold induction is depicted above the blots (n=4).
1G. MEF cells were transfected with either empty vector, EGFP-FMRP or 3×FLAG-IRE1 plasmids then pre-treated either with vehicle (dimethyl sulfoxide, DMSO) or AMG-18 (25 μM; 1 hour) followed by TG (600 nM) stimulation for 4 hours. Protein lysates were analyzed by western blotting using specific antibodies for pFMRP, FMRP, pIRE1, IRE1 and β-Actin. pFMRP/FMRP fold induction is depicted above the blots (n=4).
1H. C57BL/6 were injected either with DMSO or AMG-18 (30 mg/kg; 8 hours), followed by TM injection (1 mg/kg; 8 hours). Protein lysates of thioglycolate-elicited PM were analyzed by western blotting using antibodies for pFMRP, FMRP, pIRE1, IRE1 and β-Actin. pFMRP/FMRP fold induction is depicted above the blots (n=4).
1I. HEK293T cells were transfected with either empty vector (EV), IRE1-WT or IRE1-KD plasmids and stimulated by TG (600 nM; 1 hour). Protein lysates from each transfection were separately immunoprecipitated (IP) with anti-IRE1 antibody and subjected to a kinase reaction with purified hFMRP protein and ATP-γ-S (100 μM) in kinase buffer. The IP protein were analyzed by western blotting using specific antibodies for thiophosphate esters (ThioP), IRE1 and FMRP (n=3).
1J. Purified FMRP and IRE1 kinase (activated) proteins were subjected to kinase assay and analyzed by western blotting using specific antibodies for ThioP, IRE1 and FMRP (n=3) and with LC-MS/MS. Identified IRE1 kinase-mediated FMRP phosphorylation sites.
1K. Fmr1^{-/-} mouse embryonic fibroblasts (MEF) were transfected either with EV, WT-FMRP, SA-FMRP or STSA-FMRP plasmids followed by PA treatment (500 μM; 6 hours). Protein lysates were analyzed by western blotting using specific antibodies for FMRP, pFMRP, pIRE1 and β-Actin. pFMRP/FMRP fold induction is depicted above the blots (n=3).

FIGS. 1L-1V depict (Data are mean±SEM. Unpaired t-test with Welch's correction. * P≤0.05, ** P≤0.01, *** P≤0.001):
1L. Western blot quantifications for pFMRP/FMRP ratio in FIG. 1C.
1M. qRT-PCR analysis of Fmr1 mRNA from the samples in FIG. 1C (n=4).
1N. Western blot quantifications for pFMRP/FMRP ratio in FIG. 1D.
1O. qRT-PCR analysis for Fmr1 mRNA from the samples in FIG. 1D (n=3).
1P. Western blot quantifications for pFMRP/FMRP ratio in FIG. 1E.
1Q. Western blot quantifications for pFMRP/FMRP ratio in FIG. 1F.
1R. qRT-PCR analysis for Fmr1 mRNA from the samples in FIG. 1F (n=6).
1S. Western blot quantifications for pFMRP/FMRP ratio in FIG. 1G.
1T. Western blot quantifications for pFMRP/FMRP ratio in FIG. 1H.
1U. pFMRP sequence (SEQ ID NO: 1) covered (83%) with two different digestion strategies-Proteinase K (underlined) and Trypsin-GluC (bold) using LC-MS/MS in samples from FIG. 1H.
1V. LC-MS/MS identified phosphorylated peptides from the samples in FIG. 1H; with sites of phosphorylation (AA), charge state (z), identification parameters (XCorr and DeltaCN-greater values indicate higher confidence by peptide-spectrum match/PSM), measured precursor mass ([M+H]+), and a confidence score for localization of phosphorylation on an amino acid (A-Score, value>19 and >13, represent >99% and >95% confidence, respectively); the peptide marked with (>) was evaluated further.
SEQ ID NOS. of the sequences shown in FIG. 1V are as follows.

| Sequence | SEQ ID NO. |
|---|---|
| HLDIKENpSTHFSQPNSTK | SEQ ID NO: 2 |
| KHLDIKENpSTHFSQPNSTK | SEQ ID NO: 3 |
| KHLDIKENpSTHFSQPNSTK | SEQ ID NO: 4 |
| RGPGYTSGTNpSEASNA | SEQ ID NO: 5 |
| RGPGYTSGTNSEApSNASET | SEQ ID NO: 6 |
| ESDHRDELSDWSLAPTEEER | |
| E | |
| pSETESDHRDELSDW | SEQ ID NO: 7 |
| NAPSETESDHRDELSDW | SEQ ID NO: 8 |
| NAPSETESDHRDELSDW | SEQ ID NO: 9 |
| NAPSETESDHRDELSDW | SEQ ID NO: 10 |
| RGPGYTSGTNSEASNASEpT | SEQ ID NO: 11 |
| ESDHRDELSDWSLAPTEEER | |
| E | |
| SETEPSDHRDELSDW | SEQ ID NO: 12 |
| SVHpTKTLQNTSSEGSR | SEQ ID NO: 13 |
| SVHpTKTLQNTSSEGSR | SEQ ID NO: 14 |
| SVHpTKTLQNTSSEGSR | SEQ ID NO: 15 |
| SVHTKpTLQNTSSEGSR | SEQ ID NO: 16 |

FIGS. 2A-2M depict that FMRP deficiency enhances RCT while reducing foam cell formation in vivo. (Data are mean±SEM. Unpaired t-test with Welch's correction. * P≤0.05, ** P≤0.01, *** P≤0.001.)
2A. Fmr1^{+/+} and Fmr1^{-/-} mice were injected with AAV_PCSK9 and fed with 16 weeks of WD. Residential PM were stained with Oil Red O (ORO) and imaged (n=7; Scale bar=50 μm).
2B. Apoe-/mice were fed with WD (12 weeks) and injected with vehicle (DMSO) or AMG-18 (30 mg/kg/day) in the last 4 weeks of WD. Residential PM were stained with ORO and imaged (n=5; Scale bar=50 μm).
2C-2E. Flow cytometry analysis of BMDMs after dil-ac-LDL (25 μg/ml) loading for 24 hours; (2C) control- or Fmr1-siRNA transfected BMDM (n=4), (2D) Fmr1^{+/+} and Fmr1^{-/-} BMDM (n=4), 2(E) BMDM pre-treated with either vehicle (DMSO) or AMG-18 (5 μM; 1 hour) (n=6).
2F-2G. Macrophages were pre-loaded with fluorescently labeled cholesterol (16 hours) followed by incubation in efflux medium including APOA1 (25 μg/ml) or HDL (50 μg/ml) as acceptors for 6 hours. % Efflux was calculated as cholesterol signal in medium/cholesterol signal in medium and cell: Cholesterol efflux in (2F) Fmr1+/+ and Fmr1^{-/-} BMDM (n=4) and in (2G) BMDM that were pre-treated either with DMSO or AMG-18 (5 μM; 1 hour) (n=4).
2H-2K. RCT experiment (n=12): (2H) Schematic representation of C57BL6 mice were injected with [3H]-cholesterol-loaded foamy Fmr1^{+/+} and Fmr1^{-/-} BMDM, (2I) plasma cholesterol levels after 24 and 48 hours, (2J) liver cholesterol levels after 48 hours, and (2K) feces cholesterol levels after 48 hours (n=12).
2L. Protein lysates from macrophages used in FIG. 2C were analyzed by western blotting using specific antibodies for FMRP and β-Actin.
2M. Fmr1^{-/-} or AMG-18 pre-treated cells (10 μM, 1 hour) were treated with fluorescently labeled cholesterol for 4 hours (n=6). Data are mean±SEM. Unpaired 1-test with Welch's correction. * P≤0.05, ** P≤0.01, *** P≤0.001.

FIGS. 3A-3G depict that FMRP-deficiency increases efferocytosis in vivo. (For all images scale bar=50 μm; Red: Macrophages, Green: AC/AC-1, Violet: AC-2. Data are mean±SEM. Unpaired t-test with Welch's correction. * P≤0.05, ** P≤0.01, *** P≤0.001.)
3A-3E. In vitro and in vivo efferocytosis experiments, where percentage of macrophages F4/80⁺ (red) that ingested apoptotic cells (AC) labeled with carboxyfluorescein succinimidyl ester (CFSE)⁺ (green) were reported as % efferocytosis.
3A. BMDMs were transfected with Fmr1- or control-siRNA and incubated CFSE-labeled AC for the indicated hours (n=4).
3B. Fmr1^{+/+} and Fmr1^{-/-} BMDMs were treated with PA (500 μM) for 6 hours and then incubated with CFSE-labeled ACs for 4 hours (n=4).
3C. Fmr1^{+/+} and Fmr1^{-/-} mice were fed WD (16 weeks) and injected intraperitoneally with CFSE-labeled AC (1.5 hours), followed by PM elicitation (n=4-5).
3D. BMDM were pre-treated either with vehicle (DMSO) or AMG-18 (5 μM) for 1 hour then incubated with CFSE-labeled ACs for 4 hours (n=4).
3E. C57BL/6 mice were injected with AMG-18 (30 mg/kg) or vehicle (DMSO) for 8 hours, followed by intraperitoneal injection with CFSE-labeled ACs for 1.5 hours and PM elicitation (n=4).
3F-3G. In vitro continuous efferocytosis experiments, where macrophages were stained for F4/80⁺ (red), AC were labeled with CFSE (AC-1; green) or Violet (AC2; violet). % continuous efferocytosis was determined by the ratio of F4/80⁺, CFSE⁺ and Violet⁺ (triple positive) cells to total F4/80^{+/+} and CFSE⁺ (double positive) cells.
3F. Fmr1++ and Fmr1^{-/-} BMDM were incubated with AC-1 for 2 hours, and after 2 hours interval, incubated with AC-2 for 2 more hours (n=4-3).
3G. BMDM were pre-treated either with vehicle (DMSO) or AMG-18 (5 μM) for 1 hour, incubated with CFSE-labeled AC-1 for 2 hours, followed by incubation with Violet-labeled AC-2 for 2 hours and PM collection (n=4).

FIGS. 4A-4G depict FMRP targets in macrophages. (All results are from (n=3) independent experiments. In western blots, the fold protein expression change was calculated relative to beta-Actin and depicted above the blots and a representative blot was shown. Data are mean±SEM. Unpaired t-test with Welch's correction. * P≤0.05, ** P≤0.01, *** P≤0.001.)
4A-4B. RNA lysates from Fmr1^{+/+} and Fmr1^{-/-} BMDM that were treated with PA (500 μM; 6 hours) were fractionated using a 10%-50% sucrose gradient and separated to polysome, monosome/NTR fractions. The absorbance (260 nm) of RNA was measured and plotted as a function of time (n=3).
4A. Representative profile for RNA distribution from genotypes based on UV absorbance readings after sucrose gradient fractionation.
4B. The ratio of the Abca1, Abcg1, Mertk, Lrp1, Cd36, Cd47 and Rac1 mRNA in polysome to NTR fraction (n=3).
4C. BMDM were isolated from Fmr1^{+/+} and Fmr1^{-/-}, and protein lysates were analyzed by western blotting using specific antibodies for ABCA1, ABCG1, MerTK, LRP1, FMRP and β-Actin antibodies (n=6).
4D. Fmr1^{-/-} MEF cells were transfected with EV, WT-FMRP or STSA-FMRP plasmids followed by PA treatment (500 μM; 6 hours). Protein lysates were analyzed by western blotting using specific antibodies for ABCA1, MerTK, LRP1, pFMRP, FMRP and β-Actin (n=5).
4E. qRT-PCR analysis of Abca1, Abcg1, Mertk, Lrp1, Cd36 and Rac1 in total mRNA levels from same samples used in polysome fractions (n=3).
4F. Western blot quantifications for ABCA1, ABCG1, MerTK and LRP1 in FIG. 4C. The fold change of protein expression level was calculated relative to β-Actin.
4G. Western blot quantifications for ABCA1, MerTK and LRP1 in FIG. 4D. The fold change of protein expression level was calculated relative to β-Actin. (Data are mean±SEM. Unpaired t-test with Welch's correction. * P≤0.05, ** P≤0.01, *** P≤0.001.)

FIGS. 5A-5J depict FMRP-deficiency alleviates atherosclerosis. (Data are mean±SEM; Mann Whitney U test. * P≤0.05, ** P≤0.01, *** P≤0.001.)
5A. Atherosclerosis experiment design in Fmr1^{+/+} and Fmr1^{-/-} mice that were injected with AAV_PCSK9 and fed WD (16 weeks).
5B. Lesion area calculated from en face aorta, stained with ORO (n=12-13; Scale bar=5 mm).
5C. Total plaque area was calculated from hematoxylin & eosin (H&E)-stained aortic root sections (n=8; Scale bar=300 μm).
5D. Foam cell area was calculated from ORO-stained aortic root sections (n=8; Scale bar=300 μm).
5E. Necrotic area was calculated from H&E-stained aortic root sections (n=8; Scale bar=100 μm).
5F. Atherosclerosis experiment design in myFmr1^{+/+} and myFmr1^{-/-} mice that were injected with AAV_PCSK9 and fed WD (16 weeks).
5G. Lesion area calculated from en face aorta, stained with ORO (n=9; Scale bar=5 mm).
5H. Total plaque area was calculated from H&E-stained aortic root sections (n=9-6; Scale bar=300 μm).
5I. Foam cell area was calculated from ORO-stained aortic root sections (n=9-6; Scale bar=300 μm).
5J. Necrotic area was calculated from H&E-stained aortic root sections (n=9-6; Scale bar=100 μm).

FIGS. 5K-5T depict: (Data are mean #SEM; Mann Whitney U test. * P≤0.05, ** P≤0.01, *** P≤0.001)
5K. The ratio of mouse weight at the beginning of the experiment over the weight at the end of the experiment in FIG. 5A (n=12-13); and plasma measurements for glucose (n=12-13), cholesterol (n=12-13) and lipoproteins (n=4), respectively, from mice in FIG. 5A.
5L. Abundance of B cells, T cells and monocytes in the peripheral blood as % of total CD45+ cells from the Fmr1^{+/+} and Fmr1^{-/-} mice in FIG. 5A (n=12-13) .
5M. Macrophage area was calculated from MOMA-2 (red)-stained aortic root sections as % of MOMA2⁺ stained area to total plaque area (n=8; Scale bar=100 μm).
5N. Apoptosis was calculated from the number of TUNEL⁺ cells (green) in the MOMA-2-stained (red) plaque area (n=8; Scale bar=100 μm).
5O. Fmr1^{+/+} and Fmr1^{-/-} BMDMs or AMG-18 pre-treated cells (10 μM, 1 hour) were treated with PA (500 M) treatment for 12 hours and then stained with Propidium iodide (PI) (n=5).
5P. % Smooth muscle actin (α-SMA) was calculated from aortic root sections stained with a-SMA (green) as % α-SMA⁺ area to the total plaque area (n=8, Scale bar 100 μm).
5Q. % collagen area was calculated from Masson's Trichrome staining as the percentage of collagen (blue) area in total plaque area (n=8; Scale bar=100 μm).
5R. The ratio of mouse weight at the beginning of the experiment over the weight at the end of the experiment in FIG. 5F (n=9).
5S-5T. Plasma measurements for (5S) glucose (n=9) and (5T) cholesterol (n=9) from mice in FIG. 5F.

FIGS. 6A-6J depict that IRE1 kinase inhibition alleviates atherosclerosis. (Data are mean±SEM; Mann Whitney U test. * P≤0.05, ** P≤0.01, *** P≤0.001.)
6A. Atherosclerosis experiment design in Apoe^{-/-} mice were fed with WD (12 weeks) and injected with vehicle (DMSO) or AMG-18 (30 mg/kg) once a day in the last 4 weeks of WD.
6B. Lesion area calculated from en face aorta, stained with ORO (n=5; Scale bar=5 mm).
6C. Total plaque area was calculated from H&E-stained aortic root sections (n=5; Scale bar=300 μm).
6D. Foam cell area was calculated from ORO-stained aortic root sections (n=5; Scale bar=300 μm).
6E. Necrotic area was calculated from H&E-stained aortic root sections (n=5; Scale bar=100 μm).
6F. Atherosclerosis experiment design in Apoe^{-/-} mice were fed with WD (12 weeks) and injected with vehicle (DMSO) or AMG-18 (30 mg/kg) twice a day in the last 4 weeks of WD.
6G. Lesion area calculated from en face aorta, stained with ORO (n=6; Scale bar=5 mm).
6H. Total plaque area was calculated from H&E-stained aortic root sections (n=5; Scale bar=300 μm).
6I. Foam cell area was calculated from ORO-stained aortic root sections (n=5; Scale bar=300 μm).
6J. Necrotic area was calculated from H&E-stained aortic root sections (n=5; Scale bar=100 μm).

FIGS. 6K-6T depict: (Data are mean±SEM; Mann Whitney U test. * P≤0.05, ** P≤0.01, *** P≤0.001.)
6K. The ratio of mouse weight at the beginning of the experiment over the weight at the end of the experiment in FIG. 6A (n=5).
6L-6N. Plasma measurements for (6L) glucose (n=5) and (6M) cholesterol (n=5) from mice in FIG. 6A. (6N) Abundance of B cells, T cells and monocytes in the peripheral blood as % of total CD45+ cells from the vehicle (DMSO) or AMG-18 (30 mg/kg) once a day injected mice in FIG. 6A (n=5).
6O. Protein lysates of liver from mice in FIG. 6A. Proteins were analyzed by western blotting using specific antibodies for pIRE1 and β-Actin (n=5).
6P. The ratio of mouse weight at the beginning of the experiment over the weight at the end of the experiment in FIG. 6F (n=6).
6Q-6R. Plasma measurements for (6Q) glucose (n=6) and (6R) cholesterol (n=6) from FIG. 6F.
6S-6T. Protein lysates of (6S) thioglycolate-elicited PM and (6T) liver from mice in FIG. 6F. Proteins were analyzed by western blotting using specific antibodies for pIRE1 and β-Actin (n=6).

FIG. 7 depicts a diagram showing that IRE1-FMRP signaling controls cholesterol efflux and efferocytosis pathways in macrophages. IRE1-mediated FMRP phosphorylation suppresses translation of mRNA for key cholesterol transporters and efferocytosis receptors in macrophages and promotes atherosclerosis.

FIGS. 8A-8K depict that IRE1 kinase domain and FMRP regulates IL-1B secretion in macrophages. (Supernatants were analyzed by western blotting using specific antibody for IL-1B and caspase-1 and protein lysates were analyzed by western blotting using specific antibodies for FMRP, pIRE1, IRE1 and β-Actin.)
8A. BMDMs were transfected with Fmr1- or control-siRNA and 24 hours after transfection cells were primed with LPS (200 μM) for 3 hours followed by PA (500 μM) treatment for 16 hours (n=6).
8B. Quantification of secreted m-IL-1β to intracellular pro-IL-1β ratio normalized to β-Actin in FIG. 8A.
8C. Quantification of secreted p10 (caspase 1) to β-Actin in FIG. 9A.
8D. Fmr1^{+/+} and Fmr1^{-/-} BMDMs were primed with LPS (200 μM) for 3 hours followed by PA (500 M) treatment for 16 hours (n=6).
8E. Quantification of secreted m-IL-1β to intracellular pro-IL-1β ratio normalized to β-Actin in FIG. 8D.
8F. Quantification of secreted p10 (caspase 1) to β-Actin in FIG. 8D.
8G. The ratio of the pro-IL-1β mRNA in polysome to NTR fraction (n=3).
8H. qRT-PCR analysis of pro-IL-1β in total mRNA levels from same samples used in polysome fractions (n=3).
8I. BMDM cells were pre-treated with AMG-18 (10 μM) for 1 hour and the primed with LPS (200 μM) for 3 hours followed by PA (500 μM) treatment for 16 hours (n=6).
8J. Quantification of secreted m-IL-1β to intracellular pro-IL-1β ratio normalized to β-Actin in FIG. 8I.
8K. Quantification of secreted p10 (caspase 1) to β-Actin in FIG. 8I.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, NY 2006); March, Advanced Organic Chemistry Reactions, Mechanisms and Structure 7^{th} ed., J. Wiley & Sons (New York, NY 2013); and Sambrook and Russel, Molecular Cloning: A Laboratory Manual 4^{th} ed., Cold Spring Harbor Laboratory Press (Cold Spring Harbor, NY 2012), provide one skilled in the art with a general guide to many of the terms used in the present application. For references on how to prepare antibodies, see D. Lane, Antibodies: A Laboratory Manual 2^{nd} ed. (Cold Spring Harbor Press, Cold Spring Harbor NY, 2013); Kohler and Milstein, (1976) Eur. J. Immunol. 6:511; Queen et al. U.S. Pat. No. 5,585,089; and Riechmann et al., Nature 332:323 (1988); U.S. Pat. No. 4,946,778; Bird, Science 242:423-42 (1988); Huston et al., Proc. Natl. Acad. Sci. USA 85:5879-5883 (1988); Ward et al., Nature 334:544-54 (1989); Tomlinson I. and Holliger P. (2000) Methods Enzymol, 326, 461-479; Holliger P. (2005) Nat. Biotechnol. September; 23 (9): 1126-36).

One skilled in the art will recognize many methods and materials similar or equivalent to those described herein, which could be used in the practice of the present invention. Indeed, the present invention is in no way limited to the methods and materials described. For purposes of the present invention, the following terms are defined below.

“IRE1α” refers to a serine/threonine-protein kinase/endoribonuclease inositol-requiring enzyme 1 α, which is an enzyme that in humans is encoded by the ERN1 gene. The protein encoded by this gene is the ER to nucleus signaling 1 protein, a human homologue of the yeast Ire1 gene product. IRE1α possesses two functional enzymatic domains, an endonuclease and a trans-autophosphorylation kinase domain. Without wishing to be bound by a theory, upon activation, IRE1α oligomerizes and carries out an unconventional RNA splicing activity, removing an intron from the X-box binding protein 1 (XBP1) mRNA, and allowing it to become translated into a functional transcription factor, XBP1s.

“FMRP” refers to a protein called fragile X mental retardation protein, which in human is encoded by the FMR1 (FMRP translational regulator 1) gene.

“Efferocytosis” refers to the process by which apoptotic cells are removed by phagocytic cells, such as macrophages and other immune phagocytes.

“Foam cells” are a type of macrophage that localize to fatty deposits on blood vessel walls, where they ingest low-density lipoproteins and become laden with lipids, giving them a foamy appearance.

“Administering” and/or “administer” as used herein refer to any route for delivering a pharmaceutical composition to a patient. Routes of delivery may include non-invasive peroral (through the mouth), topical (skin), transmucosal (nasal, buccal/sublingual, vaginal, ocular and rectal) and inhalation routes, as well as parenteral routes, and other methods known in the art. Parenteral refers to a route of delivery that is generally associated with injection, including intraorbital, infusion, intraarterial, intracarotid, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders.

A therapeutically or prophylactically significant reduction in a symptom is, e.g. at least about 10%, at least about 20%, at least about 30%, at least about 40%, at least about 50%, at least about 60%, at least about 70%, at least about 80%, at least about 90%, at least about 100%, at least about 125%, at least about 150% or more in a measured parameter as compared to a control or non-treated subject or the state of the subject prior to administering the atheroprotective agent described herein.

We have made the striking discovery that Fragile X Mental Retardation Protein (FMRP) is induced by lipids in macrophages and in mouse and human atherosclerotic plaques. We found FMRP associates with and is phosphorylated by the inositol-requiring enzyme-1 (IRE1), a conserved endoplasmic reticulum (ER) stress-sensing kinase/endoribonuclease. We have identified FMRP as the first kinase substrate of IRE1 after thirty plus years since IRE1 protein was discovered as a key upstream regulator of the eukaryotic unfolded protein response. ER stress and subsequent IRE1 activation in plaques is causally associated with atherosclerosis. Enhanced IRE1 to FMRP signaling in macrophages may thus promote atherogenesis; and therefore, we conceive using inhibitors to target this signaling axis represents a new therapeutic use in preventing and treating atherosclerosis.

Our work shows FMRP inhibition leads to post-transcriptional induction of cholesterol exporters and reduces foam cell formation. Lower cholesterol levels were reported in both FMRP-deficient mice and Fragile X patients, suggesting cholesterol homeostasis is an important target for FMRP.

Herein we describe a therapeutic target for regulating atherosclerosis through the use of a small molecule inhibitor of the kinase activity of IRE1 or through targeting IRE1's substrate, FMRP. Our findings show that targeting IRE1-FMRP signaling axis affects the FMRP-regulated translational control over macrophage cholesterol trafficking and efferocytosis, which represents a new prophylactic and treatment approach to cardiovascular disease.

The IRE1-FMRP signaling pathway is induced by hyperlipidemia, and FMRP is an RBP functioning as a translational suppressor of cholesterol exporters and efferocytosis receptors in macrophages. As a result of this kinase-substrate signal transduction, efferocytosis by macrophages is reduced, and cholesterol accumulation is increased in macrophages and plaques, which promotes atherosclerotic disease progression. We have shown that disrupting this signaling axis through specific small molecule inhibitor(s) or by genetic approaches alleviates atherosclerosis in mice, and we conceive using small molecule inhibitors (such as antisense oligo for Fmr1 RNA) specifically targeting the kinase domain of IRE1, targeting FMRP, or uncoupling the kinase-substrate relationship between IRE1 and its effector FMRP, can promote reverse cholesterol transport and efferocytosis by macrophages while mitigating atherosclerosis, thereby providing prophylaxis and treatment of atherosclerosis and related cardiovascular diseases. Exemplary cardiovascular diseases include coronary artery disease, high blood pressure, cardiac arrest, congestive heart failure, arrhythmia, peripheral artery disease, stroke, and congenital heart disease.

Herein we present evidence that ER stress and hypercholesterolemia in mice induces the phosphorylation of macrophage FMRP on S500 by IRE1, a conserved ER stress-sensing kinase/endoribonuclease (RNase). Metazoans have two IRE1 paralogues, IRE1α (referred to as IRE1 below) and IRE1β. While IRE1α is ubiquitously expressed, IRE1β expression is restricted to gastrointestinal epithelium. To date, IRE1 has been described to trans-autophosphorylate as a first step in the activation of its RNase modality, which initiates a non-conventional RNA splicing reaction and the production of the transcription factor known as spliced X box protein-1 (XBP1s), one of the key drivers of the unfolded protein response (UPR). IRE1 senses both protein folding stress induced by an accumulation of unfolded proteins in the ER lumen and ER membrane lipid bilayer stress induced by an accumulation of cholesterol or saturated fatty acids (SFA). The mechanism by which IRE1 contributes to the atherosclerotic disease pathogenesis has remained elusive. Here we show that IRE1 phosphorylates FMRP on S500, which in turn leads to post-transcriptional suppression of cholesterol efflux and efferocytosis by macrophages. FMRP-deficiency and IRE1 kinase inhibition both enhance RCT and efferocytosis in vivo, reducing foam cell formation and atherosclerosis progression in mice. These findings reveal a new role for FMRP in macrophages in the regulation of cholesterol homeostasis and efferocytosis and provide mechanistic insight into IRE1-driven atherosclerotic processes during hypercholesterolemia.

Various embodiments provide methods for reducing the likelihood or inhibiting progression of atherosclerosis, or reducing the severity or treating atherosclerosis, in a subject in need thereof, which comprise administering to the subject a composition comprising a therapeutically effective amount of an inhibitor of inositol-requiring enzyme-1 (IRE1), an inhibitor of fragile X mental retardation protein (FMRP), or both.

Further embodiments provide methods for enhancing macrophage's activity in engulfing apoptotic cells for clearance, exporting cholesterol to lipid-poor apolipoprotein, and/or internalizing low density lipoprotein (LDL), reducing the likelihood of foam cell formation from the macrophage, and/or reducing atherosclerotic lesion or necrotic core area of the atherosclerotic lesion in a blood vessel, said atherosclerotic lesion comprising the macrophage, wherein the methods comprise contacting the macrophage with an inhibitor of IRE1, an inhibitor of FMRP, or both; or transfecting or expressing a vector encoding the inhibitor of IRE1, the inhibitor of FMRP, or both in the macrophage. In some aspects, the macrophage is exposed or has been exposed to a stressor such as a saturated fatty acid, an oxidized low density lipoprotein (oxLDL), thapsigargin, or tunicamycin. In some implementations, the methods for enhancing the activity of macrophage results in reduction of atherosclerotic lesion in en face preparation of the blood vessel.

In some embodiments, the inhibitor of IRE1 is a small molecule that inhibits kinase activity of the IRE1, wherein in the presence of the IRE1 and the inhibitor of IRE1, phosphorylation of FMRP is reduced compared to that in the presence of the IRE1 without the inhibitor of IRE1. In some embodiments, the inhibitor of IRE1 for use in the disclosed prophylactic or treatment methods is an inhibitor targeting the ATP-binding pocket of the kinase domain of IRE1α. In some embodiments, the small molecule inhibitor for the disclosed use herein is not an inhibitor of IRE1's endoribonuclease (RNase) domain. Exemplary inhibitors that inhibit the kinase activity of IRE1 or target the ATP-binding pocket of the kinase domain are AMG-18, sunitinib, APY29, compound 3. In some aspects, the IRE1 inhibitor comprises AMG-18.

Yet in some aspects, the IRE1 inhibitor is selected from AMG-18, methotrexate, cefoperazone, folinic acid, fludarabine phosphate, ORIN1001, G-9807, G-1749, STF-083010, 4μ8C, salicylaldehydes, MKC-3946, MCK8866, or toyocamycin.

Salicylaldehydes, MKC-3946, toyocamycin, STF-083010 and 4μ8C are small molecules that selectively inhibit IRE1's RNase function. Therefore, in other implementations, the treatment methods provided herein does not include administering salicylaldehydes, MKC-3946, toyocamycin, STF-083010, or 4μ8C to a subject in need thereof.

In some embodiments, the inhibitor of IRE1 is a small interfering RNA (siRNA), microRNA (miRNA), or antisense oligo (ASO) against a gene encoding Ire1 (or more specifically against ERN1, the human gene that encodes human IRE1α, or against Ern1, the mouse gene that encodes mouse IRE1α), thereby silencing the gene (such as mRNA) or suppressing expression of IRE1.

An exemplary siRNA against Ern1 is SI0099588 from Qiagen, which is described in geneglobe.qiagen.com/us/search/?cat=&q=SI0099588% 20&pgid=flexitube-sirna. For example, the inhibitor of IRE1 is a siRNA, miRNA, or ASO against at least a portion or full sequence of ERN1 mRNA (e.g., NCBI reference sequence: NM_001433.5) or Ern1 mRNA (e.g., NCBI reference sequence: NM_023913). In some implementations, the siRNA, miRNA, or antisense oligo is against about 1%, 2%, 3%, 4%, 5%, 6%, 7%, 8%, 9%, 10%, 20%, 30%, 40%, 50%, 60%, 70%, 80%, 90%, or a range in between any of the two numbers, of ERN1 mRNA or Ern1 mRNA in base pair numbers or base numbers, resulting in suppression of expression of at least the kinase domain of the gene.

In some embodiments, the inhibitor of FMRP is an siRNA, miRNA, or antisense oligo against FMR1, the human gene that codes for FMRP protein, or against Fmr1, the mouse gene that codes for FMRP protein, thereby silencing the gene or suppressing expression of FMRP protein.

An exemplary siRNA against Fmr1 is AM16708 from Thermofisher Scientific, which is described in www.thermofisher.com/order/genome-database/details/sirna/157567?CID=&ICID=&subtype=. For example, the inhibitor of FMRP is a siRNA, miRNA, or ASO against at least a portion or full sequence of FMR1 mRNA (e.g., UniProt Q06787) or Fmr1 mRNA (e.g., UniProt P35922; NCBI reference sequence NM_001290424 or NM_008031; or GenBank AF461114, AK046605, AK053211, AK053701, AK053829, AK080948, AK140343, or BC079671). In some implementations, the siRNA, miRNA, or antisense oligo is against about 1%, 2%, 3%, 4%, 5%, 6%, 7%, 8%, 9%, 10%, 20%, 30%, 40%, 50%, 60%, 70%, 80%, 90%, or a range in between any of the two numbers, of FMR1 mRNA or Fimr1 mRNA in base pair numbers or base numbers, resulting in suppression of expression of at least the kinase domain of the gene.

An exemplary siRNA against Fmr1 (ref seq: NM_008031) has a sense strand sequence of GCAUGUGAUGCUACGUAUATT (SEQ ID NO: 17), and an antisense strand sequence of UAUACGUAGCAUCACAUGCTG (SEQ ID NO: 18).

Another exemplary siRNA against Fmr1 (ref seq: NM_008031) has a sense strand sequence of GGUGCCAGAAGAUUUACGATT (SEQ ID NO: 19), and an antisense strand sequence of UCGUAAAUCUUCUGGCACCTC (SEQ ID NO: 20).

In some embodiments, the siRNA molecule has a duplex region and either no overhang regions or at least one overhang region, (each overhang region containing six or fewer nucleotides,) wherein the duplex region consists of a sense region (a.k.a. passenger strand) and an antisense region (a.k.a. guide strand), wherein said sense region and said antisense region together form said duplex region and said antisense region and said sense region each 19-30 nucleotides in length and said antisense region comprises a sequence that is the reverse complement of the intended target mRNA. Alternatively speaking, the sense region comprises a sequence that is the same sequence as the target mRNA. Small double-strand siRNAs are transfected into cells where the guide strand is loaded into RISC; and transfection can be realized with cationic lipid or polymer-based transfection reagents, or via electroporation. This activated protein and nucleic acid complex can then elicit gene silencing by binding, through perfect complementarity, to a single target mRNA sequence, thereby targeting it for cleavage and degradation.

In some embodiments, the ASO is a locked nucleic acid (LNA)-enhanced antisense oligonucleotide, such as antisense LNA GapmeRs from Qiagen which is single-stranded ASO in a LNA form (shown below) of RNA analogs for silencing of lncRNA and mRNA. For example, a LNA-enhanced ASO against the target has a sequence from 5′ to 3: GGAATAAGAATTACGG (SEQ ID NO: 21).

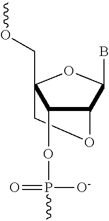

Structure of LNA: the ribose ring is connected by a methylene bridge between the 2′-O and 4′-C atoms, “locking” the ribose ring in the ideal conformation for Watson-Crick binding. When incorporated into a DNA or RNA oligonucleotide, LNA makes the pairing with a complementary nucleotide strand more rapid and increases the stability of the resulting duplex.

In yet additional embodiments, the inhibitor of IRE1 can be pharmacological compounds that inhibit human IRE1 activity or decreases IRE1-mediated unfolded protein response (UPR) activity, as disclosed in U.S. Pat. Nos. 8,697,709 and 8,980,899, which are incorporated by reference herein in their entirety. For example, the pharmacological compound has a formula:

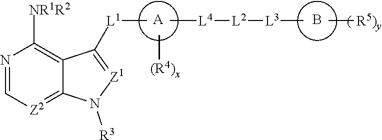

wherein x is an integer from 0 to 4; y is an integer from 0 to 5; ring A is arylene or heteroarylene; ring B is aryl or heteroaryl, Z¹ is —N═ or —C(R²²)═, Z² is —N═ or —C(R²³)═, R¹ and R² are independently hydrogen, substituted or unsubstituted alkyl, or substituted or unsubstituted heteroalkyl; R³ is independently —CN, —CF₃, —S(O)ₙR⁶, —N(O)ₘ, —NR⁷R⁸, —C(O)R⁹, —NR¹⁰—C(O)R¹¹, —NR¹²—C(O)—OR¹³, —C(O)NR¹⁴R¹⁵, —NR¹⁶(O)₂R¹⁷, —S(O)₂NR¹⁸R¹⁸′, —OR¹⁹, halomethyl, unsubstituted alkyl, unsubstituted heteroalkyl, unsubstituted cycloalkyl, unsubstituted aryl, or unsubstituted heteroaryl; R²² and R²³ are independently —CN, —CF₃, —S(O)ₙR⁶, —N(O)ₘ, —NR⁷R⁸, —C(O)R⁹, —NR¹⁰—C(O)R¹¹, —NR¹²—C(O)OR¹³, —C(O)NR¹⁴R¹⁵, —NR¹⁶S(O)₂R¹⁷, —S(O)₂NR¹⁸R¹⁸′, —OR¹⁹, halomethyl, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁴ and R⁵ are independently halogen, —CN, —CF₃, —S(O)ₙR⁶, —N(O)ₘ, —NR⁷R⁸, —C(O)R⁹, —NR¹⁰—C(O)R¹¹, —NR¹²—C(O)—OR¹³, —C(O)NR¹⁴R¹⁵, —NR¹⁶S(O)₂R¹⁷, —S(O)₂NR¹⁸R¹⁸, —OR¹⁹, halomethyl, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; n is an integer from 0 to 2; m is an integer from 1 to 2; R⁶, R⁷, R⁸, R⁹, R¹⁰, R¹¹, R¹², R¹³, R¹⁴, R¹⁵, R¹⁶, R¹⁷, R¹⁸, R¹⁸′, and R¹⁹ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; L¹ is a bond, substituted or unsubstituted alkylene or substituted or unsubstituted heteroalkylene; L² is —C(O)—; L³ is —NH—; and L⁴ is —NH—.

Further embodiments provide that the inhibitor of IRE1 or the inhibitor of FMRP is an antibody, which blocks or neutralizes the kinase activity domain of IRE1 or the binding domain of FMRP with IRE1. In another embodiment, the inhibitor is a phosphatase, such that phosphorylation of FMRP is inhibited or reduced. In yet another embodiment, the inhibitor is a kinase inhibitor, such that IRE1 is inhibited or deterred from inducing phosphorylation of FMRP.

In some embodiments, the subject is experiencing ER stress in the macrophage or has been exposed to hyperlipidemia or hypercholestorelimia. In some embodiments, the macrophage is stressed by a saturated fatty acid, an oxidized low density lipoprotein (oxLDL), thapsigargin, or tunicamycin, and the IRE1 inhibitor or the FMRP inhibitor protects the macrophage from becoming a foam cell, or maintains or elevates the macrophage's ability in engulfing apoptotic cells for clearance (efferocytosis), exporting cholesterol (efflux of cholesterol to lipid-poor apolipoprotein), and/or internalizing low density lipoprotein, in the presence of the stressor.

In some embodiments, a subject in need of a prophylactic or therapeutic treatment disclosed herein is one who has atherosclerosis. In some embodiments, a subject in need of a prophylactic or therapeutic treatment disclosed herein is one who is at risk of developing atherosclerosis, e.g., having an increased level of cholesterol in blood compared to a normal or healthy person, having abdominal obesity, diabetes, high alcohol intake, high blood pressure, or not eating fruits and vegetables, not exercising regularly, smoking, and stress. In some embodiments, a subject in need of a prophylactic or therapeutic treatment disclosed herein is one who has a cardiovascular disease. In some embodiments, a subject in need of a prophylactic or therapeutic treatment disclosed herein is one who has obesity. In some embodiments, a subject in need of a prophylactic or therapeutic treatment disclosed herein is one with hyperlipidemia, hypercholestorelimia, and/or atherosclerosis. In further embodiments, a subject in need of a prophylactic or therapeutic treatment disclosed herein is one with hypercholesterolemia-induced atherosclerosis. In yet another embodiment, a subject in need thereof is one undergoing high fat diet or having been exposed to high fat diet.

Some embodiments provide that the prophylactic or treatment methods herein further include selecting a subject diagnosed with or suffering from atherosclerosis, obesity, hyperlipidemia, hypercholestorelimia, or another-related cardiovascular disease. Further embodiments provide that the subject is not diagnosed with, or does not suffer from, fragile X mental retardation syndrome.

In some embodiments, a therapeutically effective amount of the inhibitor disclosed herein for providing prophylactic or treatment benefits in a subject in need thereof includes one or more doses, wherein a dose is in the range of about 10-50 mg, 50-100 mg, 100-150 mg, 150-200 mg, 100-200 mg, 200-300 mg, 300-400 mg, 400-500 mg, 500-600 mg, 600-700 mg, 700-800 mg, 800-900 mg, 900-1000 mg, 1000-1100 mg, 1100-1200 mg, 1200-1300 mg, 1300-1400 mg, 1400-1500 mg, 1500-1600 mg, 1600-1700 mg, 1700-1800 mg, 1800-1900 mg, 1900-2000 mg, 2000-2100 mg, 2100-2200 mg, 2200-2300 mg, 2300-2400 mg, 2400-2500 mg, 2500-2600 mg, 2600-2700 mg, 2700-2800 mg, 2800-2900 mg or 2900-3000 mg.

In further embodiments, a therapeutically effective amount of the inhibitor disclosed herein for providing prophylactic or treatment benefits in a subject in need thereof includes one or more doses, wherein a dose is in the range of 0.001-0.005 mg/kg, 0.005-0.01 mg/kg, 0.01-0.02 mg/kg, 0.02-0.04 mg/kg, 0.04-0.06 mg/kg, 0.06-0.08 mg/kg, 0.08-1 mg/kg, 1-5 mg/kg, 5-10 mg/kg, 10-15 mg/kg, 15-20 mg/kg, 20-25 mg/kg, 25-30 mg/kg, 30-35 mg/kg, 35-40 mg/kg, 40-45 mg/kg, 45-50 mg/kg, 10-50 mg/kg, 50-100 mg/kg, 100-150 mg/kg, 150-200 mg/kg, 100-200 mg/kg, 200-300 mg/kg, 300-400 mg/kg, 400-500 mg/kg, 500-600 mg/kg, 600-700 mg/kg, 700-800 mg/kg, 800-900 mg/kg, 900-1000 mg/kg, 1000-1100 mg/kg, 1100-1200 mg/kg, 1200-1300 mg/kg, 1300-1400 mg/kg, 1400-1500 mg/kg, 1500-1600 mg/kg, 1600-1700 mg/kg, 1700-1800 mg/kg, 1800-1900 mg/kg, 1900-2000 mg/kg, 2000-2100 mg/kg, 2100-2200 mg/kg, 2200-2300 mg/kg, 2300-2400 mg/kg, 2400-2500 mg/kg, 2500-2600 mg/kg, 2600-2700 mg/kg, 2700-2800 mg/kg, 2800-2900 mg/kg or 2900-3000 mg/kg.

In some embodiments, a therapeutically effective amount includes two, three, or more doses administered on a daily, weekly, biweekly, monthly, quarterly, or yearly frequency; or continued when evaluation of the severity of the disease shows improvement, compared to before the last dose, until the disease is successfully treated or the subject shows no symptoms or signs of the disease.

In other embodiments, the inhibitors disclosed herein are administered using a controlled (sustained) release formulation. In particular embodiments, an inhibitor is suspended in a sustained-release matrix, made of materials, usually polymers, which are degradable by enzymatic or acid-base hydrolysis or by dissolution. Exemplary controlled or sustained release formulation is a gel-based formulation based on poly(ε-caprolactone-PEG)-b-poly(ε-caprolactone) multiblock-copolymer (a biodegradable implant); an octreotide subcutaneous (s.c.) depot (Novartis Pharma AG, Basel, Switzerland; a liquid solution based on naturally occurring lipids, which upon injection absorbs interstitial aqueous fluid, resulting in in situ transformation from liquid into a highly viscous liquid-crystal gel phase). Other controlled or sustained release formulation is described by A. Patel et al., Ther Deliv., 2014 March; 5 (3): 337-65, which is incorporated by reference herein in its entirely.

Pharmaceutical compositions including the one or more inhibitors disclosed herein may further include a pharmaceutical carrier, diluent, or excipient and optionally other medicinal agents, carriers, adjuvants, diluents, tissue permeation enhancers, solubilizers, and the like.

In some implementations, the prophylactic and treatment methods further include administering an antiplatelet medicine to the subject.

Various methods may be utilized to administer the inhibitor or atheroprotective agent disclosed or identified by methods herein, including but not limited to oral, intraperitoneally, intravenously, transmucosal, bucally, transdermal, parenteral, intra-cecal, aerosol, nasal (by inhalation), ocularly (such as intraocularly), intracisternally, intravaginally, intrarectally, implantable pump, continuous infusion, topical application (e.g., by powder, ointments, drops, suppository, or transdermal patch), capsules and/or injections.

Additional embodiments provide methods for screening for an atheroprotective agent that inhibits kinase activity of IRE1, inhibits binding between IRE1 and FMRP, or reduces phosphorylation of FMRP, and the methods comprise contacting a molecule of interest with an atherosclerotic lesion cell type, said cell type expressing IRE1 and FMRP, and measuring an amount of phosphorylation of the FMRP in the cell type, wherein a decrease in the amount of phosphorylation of the FMRP relative to an amount before the contact with the molecule of interest is indicative that the molecule of interest is an atheroprotective agent, and wherein the atherosclerotic lesion cell type comprises macrophage, endothelial cell, or vascular smooth muscle cell.

In some aspects, the atherosclerotic lesion cell type is macrophage. Other atherosclerotic lesion cell types besides macrophages include endothelial cells and vascular smooth muscle cells. Further embodiments provide that the method includes contacting the small molecule inhibitor with endothelial cells and/or vascular smooth muscle cells in an atherosclerotic lesion. Other embodiments provide that the method includes contacting the small molecule inhibitor with macrophages in an atherosclerotic lesion, rather than endothelial cells or vascular smooth muscle cells.

In some implementations of the screening methods, an additional step is contacting the atherosclerotic lesion cell type with a stressor, such that the molecule of interest decreases the amount of phosphorylation of the FMRP even in the presence or under the influence of the stressor.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

Fragile X Mental Retardation protein (FMRP), widely known for its role in hereditary intellectual disability, is an RNA-binding protein (RBP) that controls translation of select mRNAs. We discovered that endoplasmic reticulum (ER) stress induces phosphorylation of FMRP on a site that is known to enhance translation inhibition of FMRP-bound mRNAs. We show ER stress-induced activation of Inositol requiring enzyme-1 (IRE1), an ER-resident stress-sensing kinase/endoribonuclease, leads to FMRP phosphorylation and to suppression of macrophage cholesterol efflux and apoptotic cell clearance (efferocytosis). Conversely, FMRP-deficiency and pharmacological inhibition of IRE1 kinase activity enhances cholesterol efflux and efferocytosis, reducing atherosclerosis in mice. Our results provide mechanistic insights into how ER stress-induced IRE1 kinase activity contributes to macrophage cholesterol homeostasis and indicate IRE1 inhibition as a promising new way to counteract atherosclerosis.

### Lipids Induce FMRP Phosphorylation in IRE1 Kinase-Dependent Manner.

Multiple FMRP-interacting proteins potentially associates with IRE1 (FIG. 1A) based on previous mass spectrometry-based IRE1 interactome data. These proteins share significant sequence homology with FMRP and are usually found in homo/heteromeric complexes with FMRP. Based on these observations, we reasoned that FMRP might also physically interact with IRE1. Indeed, FMRP co-immunoprecipitated with IRE1 in non-stress and ER stress conditions induced by thapsigargin (TG; an inhibitor of the ER Ca²⁺ pump) and tunicamycin (TM; an inhibitor of N-linked glycosylation) from human embryonic kidney cell line (HEK293T) cells that were transiently transfected with plasmids encoding both proteins (FIG. 1B).

Since FMRP phosphorylation is important for translation suppression and the association with IRE1 juxtaposes it to a kinase whose substrate is unclear, we wondered whether ER stress alters FMRP phosphorylation state. To assess this possibility, we treated cultured macrophages with known ER stressors, such as TG and oxidized low density lipoprotein (oxLDL, another inducer of ER stress). Using specific antibodies that recognize S724 phosphorylation on IRE1 and S500 phosphorylation on FMRP, we found that TG and oxLDL significantly induced IRE1 autophosphorylation and FMRP phosphorylation but did not affect levels of FMRP protein or Fmr1 mRNA (FIGS. 1C and 1L, 1M). Phosphatase treatment of the samples partially reversed the ER stress-induced increase in the pFMRP/FMRP ratio (FIG. 1C, IM), implying FMRP phosphorylation is enhanced by these ER stressors.

We next investigated whether hyperlipidemia also has an effect on FMRP phosphorylation on S499 (mouse S499 corresponds to human S500). To this end, we used mice deficient in apolipoprotein E (Apoe^{-/-}) as this is a protein found in plasma lipoprotein particles and facilitates cholesterol clearance from the circulation. We observed that ER stress (as monitored by IRE1 autophosphorylation) was induced in the peritoneal macrophages (PM) obtained from hyperlipidemic, Apoe^{-/-} mice that were fed with a Western diet (WD) for 16 weeks when compared to Apoe^{-/-} mice fed with chow diet (CD) (FIG. 1D). We found that FMRP S499 phosphorylation, which leads to FMRP-mediated translational suppression, was 1.4-fold elevated by a chronic exposure to hypercholesterolemia, whereas FMRP protein and Fmr1 mRNA expression levels remained unchanged (FIG. 1D; 1N, 1O).

We next asked what role IRE1 plays in ER stress-induced FMRP phosphorylation. To address this question, we transfected IRE1-specific siRNA to suppress IRE1 expression in a human cell line, followed by ER stress induction by a saturated fatty acid, palmitate (PA), that is known to induce ER stress, or TG. While both ER stressors induced the phosphorylation of IRE1 and FMRP, this was prevented in IRE1 knock-down cells (FIGS. 1E and 1P). In addition, we analyzed hypercholesterolemia-induced FMRP phosphorylation in PMs obtained from Apoe^{-/-} mice with a genetic deletion of IRE1α in the myeloid lineage (IRE1^{-/-}) after feeding with WD for 16 weeks. FMRP phosphorylation, but not FMRP protein or Fmr1 mRNA, was reduced in PM isolated from IRE1^{-/-} mice when compared to those isolated from IRE1/mice (FIG. 1F, 1Q, 1R). While these results show a clear reduction of FMRP phosphorylation upon siRNA treatment or gene knock-out, we observed residual signal in both cases (FIGS. 1E and 1F). As we confirm below (FIG. 1K), the antibody used in these experiments is phosphorylation specific. We therefore surmise that partial phosphorylation on S500/S499 may also be mediated by the other kinases that are known to phosphorylate this residue on FMRP, in addition to IRE1 as we show here. Collectively, our in vitro and in vivo results strongly support that ER stress-induced activation of IRE1 kinase leads to enhanced phosphorylation of FMRP on S500/S499.

### IRE1 Phosphorylates FMRP.

To begin investigating the role of IRE1's kinase activity in ER stress-induced FMRP phosphorylation, we expressed a 3×FLAG-tagged IRE1 (FLAG-IRE1) and/or EGFP-FMRP in wild type mouse embryonic fibroblasts (MEFs). Endogenous FMRP and IRE1 migrated faster in SDS-PAGE gel than the epitope tagged EGFP-FMRP and FLAG-IRE1, respectively. In all conditions, the MEFs were TG-treated (to stimulate IRE1 kinase activity) in the absence or presence of an IRE1 kinase-specific inhibitor (AMG-18). In the absence of AMG-18, both IRE1 and FMRP were phosphorylated. AMG-18 treatment prevented IRE1 phosphorylation while clearly reducing FMRP phosphorylation (FIGS. 1G and 1S). While this data further supports the notion that IRE1 phosphorylates FMRP during ER stress, alternative kinase(s) appears to mediate the same reaction. This is consistent the published data that several kinases can phosphorylate the same residue on FMRP. Furthermore, in an in vivo setting where mice were injected with TM to induce IRE1 kinase activity, treatment with AMG-18 inhibited IRE1 kinase activity and reduced FMRP phosphorylation in PM (FIGS. 1H and 1T). Taken together, our findings support the notion that IRE1 kinase activity makes an important contribution to ER stress-induced FMRP S499 phosphorylation in mouse macrophages and MEFs as well as S500 in HEK293T cells. Our data also supports that other known or unknown FMRP kinase(s) are responsible for basal FMRP phosphorylation that is observed in non-stress conditions.

To ask whether IRE1 can phosphorylate FMRP directly, we employed in vitro assays. To this end, we immunoprecipitated wild type (WT) or kinase dead (KD) mutant IRE1 from HEK293T cells (after treating with ER stressor) and incubated the immunoprecipitates with purified, recombinant human FMRP protein in a kinase reaction. The reaction included ATP-γ-S instead of ATP, which allows kinases to thio-phosphorylate their substrates. The resultant kinase reaction was analyzed by Western blotting using an anti-thiophosphate ester antibody. In the reaction containing IRE1-WT, both proteins were thio-phosphorylated, but not in the reaction containing the IRE1-KD mutant (FIG. 1I).

To determine the specific amino acids phosphorylated by IRE1, we performed the kinase reaction using purified, recombinant human IRE1-kinase/RNase domains (amino acids 468-977) and human FMRP proteins. Both IRE1 and FMRP were phosphorylated in this reaction in an AMG-18-sensitive manner (FIG. 1J). Liquid chromatography-mass spectrometry (LC-MS/MS)-based analysis of the phosphorylated FMRP residues in this kinase reaction revealed eight IRE1-induced phosphorylation sites on FMRP at serine (S362, S494, S497, S500, S504) and threonine (T502, T592, T594) (FIG. 1J, 1U, 1V). Importantly, S500 in human FMRP is the previously identified FMRP phosphorylation site that was shown to enhance FMRP-mediated translational suppression.

Using site-directed mutagenesis, we engineered two mutant versions of FMRP, in which we either changed S500 to alanine (SA mutant) or S500, T502, and S504 to alanine (STSA triple mutant) to block phosphorylation at S500 and amino acids in its proximity. We then induced ER stress with PA in Fmr1^{-/-} MEFs transiently transfected with WT FMRP and FMRP mutants (SA and STSA). PA induced FMRP phosphorylation in WT FMRP-reconstituted cells but failed to do so in cells expressing the SA and STSA mutants of FMRP (FIG. 1J). These data confirm the specificity of the FMRP antibody for phosphorylated S500 and is consistent with the notion that IRE1 kinase directly phosphorylates human FMRP protein on S500.

IRE1-FMRP Signaling Induces Foam Cell Formation while Suppressing RCT.

We next looked into the role that FMRP plays in macrophage biology that is relevant to atherosclerotic plaque development. We performed an in vivo macrophage foam cell formation assay in the peritoneum of Fmr1^{-/-} and Fmr1^{-/-} mice, using a well-established method in which we induced hyperlipidemia using a combination of adenoviral-delivery of proprotein convertase subtilisin kexin 9 (AAV_PCSK9), a protein that directs hepatic low density lipoprotein (LDL) receptors for degradation, and feeding with WD for 16 weeks. FMRP-deficiency significantly reduced foam cell formation in vivo (FIG. 2A). Next, we fed Apoe^{-/-} mice with a WD (12 weeks) and injected them daily with the IRE1 kinase inhibitor, AMG-18, or vehicle for the last 4 weeks. AMG-18 reduced foam cell formation in the peritoneum in vivo (FIG. 2B). This result indicates that IRE1-FMRP signaling axis enhances foam cell formation.

We also transfected wild type bone marrow-derived macrophages (BMDMs) with either Fmr1-specific or control siRNA and followed by loading the cells with 3,3′-dioctadecylindocarbocyanine (dil)-labeled acetylated LDL (Ac-LDL). Flow cytometry analysis revealed that Fmr1 silencing significantly reduced % dil-acLDL internalized in macrophages (FIG. 2C, 2L). Likewise, Fmr1^{-/-} BMDM displayed reduced % dil-acLDL internalization when compared to Fmr1/BMDM (FIG. 2D). A similar reduction in foam cell formation was observed with the IRE1 kinase inhibitor, AMG-18 (FIG. 2E). These results indicate that both the inhibition of IRE1 kinase activity and the genetic deletion of its proposed substrate, FMRP, reduce foam cell formation in vitro and in vivo. Reduced foam cell formation could be explained with less cholesterol uptake, however, neither FMRP knock down nor IRE1 kinase inhibition altered cholesterol uptake in macrophages (FIG. 2M). We reasoned that this observation is most likely related to an increase in cholesterol export (RCT; due to increased translation of cholesterol exporters) from Fmr1^{-/-} macrophages. Indeed, FMRP deficiency led to an increase in cholesterol efflux coupled to its loading onto the cholesterol carriers, apolipoprotein-A1 (APOA1) and HDL (FIG. 2F), and, likewise, the IRE1 kinase inhibitor enhanced cholesterol efflux (FIG. 2G). Thus, ER stress-associated reduction in cholesterol efflux is dependent on both IRE1 kinase activity and FMRP.

Next, we determined whether the absence of FMRP in BMDMs also enhances RCT in vivo. To this end, we pre-loaded Fmr1^{+/+} and Fmr1^{-/-} BMDMs with [H3]-cholesterol and injected the cells subcutaneously into WT mice (FIG. 2H). FMRP-deficiency in macrophages significantly increased radioactivity counts ([3H]-cholesterol) in the plasma, liver, and feces of the recipient mice, when compared to recipient mice that received Fmr1/macrophages (FIG. 2I-2K), demonstrating that FMRP-deficiency in macrophages enhances RCT in mice.

### FMRP Regulates Macrophage Efferocytosis.

We next investigated the impact of FMRP-deficiency on efferocytosis, a primary process that promotes atherosclerotic plaque regression by removing apoptotic macrophages in the lesion area. To this end, we transfected BMDM (red fluorescent-stained) with Fmr1-specific or control siRNA and incubated them with green fluorescent-labeled apoptotic cells (ACs), in which apoptosis was induced by ultraviolet (UV) irradiation. FMRP knock-down increased efferocytosis of ACs (as measured by colocalization of the fluorescent markers), when compared to control (FIG. 3A). FMRP-deficient BMDMs also increased efferocytosis when compared to wild type BMDMs under both no-stress and PA-induced ER stress conditions (FIG. 3B). Next, we induced hyperlipidemia in Fmr1^{-/-} and Fmr1^{-/-} mice as described above using a combination of AAV_PCSK9 injection and feeding with a WD. We then injected the mice intraperitoneally with green fluorescent-labeled ACs and harvested PM macrophages. FMRP-deficient PMs displayed enhanced efferocytosis compared to WT PMs (FIG. 3C).

We next asked how IRE1 kinase activity impacts efferocytosis by macrophages. We observed that AMG-18 increased efferocytosis of ACs in BMDMs (FIG. 3D). Next, we injected wild type mice with AMG-18 (for 8 hours) followed by injection of green fluorescent-labeled ACs. AMG-18 also induced efferocytosis of ACs by PM in vivo (FIG. 3E).

Continued clearance of ACs by macrophages prevents the accumulation of necrotic cells and is an important process that promotes atherosclerosis regression. To determine whether Fmr1^{-/-} macrophages can efficiently internalize multiple ACs over consecutive rounds of engulfment, we incubated macrophages with green fluorescent-labeled AC for 2 hours, followed by second incubation with violet fluorescent-labeled AC for 2 more hours. FMRP-deficient BMDMs displayed an increase in continued efferocytosis when compared to wild type BMDMs (FIG. 3F). Likewise, treatment of wild type BMDM with AMG-18 enhanced continued efferocytosis of ACs (FIG. 3G). Collectively, these results demonstrate that the ablation of IRE1 kinase activity and its proposed substrate, FMRP, enhances efferocytosis in vitro and in vivo.

### Translational Suppression of Cholesterol Transporters and Efferocytosis Regulators by FMRP During ER Stress.

Consistent with our observations that macrophage FMRP plays a role in suppressing cholesterol efflux and efferocytosis, previous data describing the FMRP-RNA interactome also indicates that FMRP interacts with mRNAs encoding proteins involved in cholesterol trafficking (such as Abca1 and Abcg1) and efferocytosis receptors (such as c-Mer tyrosine kinase (Mertk) and LDL receptor-related protein 1 (Lrp1)), indicating FMRP may impair their translation. To assess this notion in macrophages, we next performed polyribosome profiling in Fmr1^{-/-} and Fmr1^{-/-} BMDMs under PA-induced ER stress conditions (FIGS. 4A and 4B). Indeed, the mRNA abundance for the cholesterol transporters, Abca1 and Abcg1, and the mRNA abundance for efferocytosis regulators, Mertk, Lrp1, and Cd36, were increased in translating polysome fractions and decreased in non-translating (NTR) fractions in the Fmr1^{-/-} BMDMs when compared to Fmr1^{-/-} BMDMs (FIG. 4B), while the abundance of these mRNAs in the total cell lysate was unchanged (FIG. 4E).

We next analyzed the corresponding protein expression changes for the FMRP-regulated mRNA targets from the same experiment in FIGS. 4A and 4B. As expected, ABCA1, ABCG1, MerTK, and LRP1 protein expression levels were induced in Fmr1^{-/-} BMDMs (FIGS. 4C and 4F). To further assess the impact of IRE1-mediated FMRP phosphorylation on the translation of FMRP's targets, we overexpressed the FMRP phosphorylation-deficient mutant (STSA) or WT-FMRP in Fmr1^{-/-} MEFs and treated with PA to induce ER stress. ABCA1, LRP1 and MerTK expression levels were reduced in the Fmr1^{-/-} MEF expressing WT-FMRP, but not in Fmr1^{-/-} MEFs expressing STSA-FMRP (FIGS. 4D and 4G). Taken together, our data demonstrate that IRE1-mediated FMRP phosphorylation tunes cholesterol transporters and efferocytosis regulators expression in macrophages.

### FMRP Knock-Down and IRE1 Kinase Inhibition Alleviates Atherosclerosis.

Our findings demonstrate that both IRE1 kinase inhibition and FMRP-deficiency result in increased RCT, reduced foam cell formation, and enhanced efferocytosis in vivo, indicating that FMRP-deficiency in mice leads to protection from atherosclerosis. We tested this notion using Fmr1^{-/-} and Fmr1^{-/-} mice in which hyperlipidemia was induced by combining AAV_PCSK9 injection with a WD as described above (FIG. 5A). Although there was a very slight decrease in body weight ratio; the plasma glucose, total plasma cholesterol (TPC), lipoprotein levels, and the number of circulating, major type of immune cells were indistinguishable between the Fmr1^{-/-} and Fmr1^{-/-} genotypes (FIG. 5K, 5L). Yet, FMRP-deficiency resulted in significant reduction in atherosclerotic lesions in en face aorta preparations (FIG. 5B). (Note: En face preparations are made by cutting a vessel longitudinally along its entire length.) FMRP-deficiency did not alter aortic root lesion area despite a significantly decreased foam cell area (as assessed by Oil Red O staining) (FIGS. 5C and 5D). We next asked what may be contributing to this phenotype. The necrotic core area in the lesions from Fmr1^{-/-} mice was significantly less than in Fmr1^{-/-} lesions (FIG. 5E), indicating improved AC clearance by Fmr1^{-/-} macrophages in plaques. We also observed a significant reduction in lesion macrophages (as assessed by the anti-monocyte macrophage 2 (MOMA-2)-stained area) and the number of apoptotic cells (TUNEL-stained) per macrophage area (FIG. 5M, 5N). We further investigated whether apoptosis is altered in Fmr1^{-/-} and AMG-18 treated macrophages. There was no significant change between the groups (FIG. 5O), supporting the notion that the primary consequence of inhibiting IRE1-FMRP signaling is efficient clearance of apoptotic cells through increasing efferocytosis capacity. Additionally, we detected no change in smooth muscle area (stained with smooth muscle actin (SMA)), but there was a significant increase in the collagen content of Fmr1^{-/-} lesions when compared to Fmr1^{-/-} lesions, indicating this could be the reason why aortic root lesion area is not significantly altered (FIG. 5P, 5Q).

To approach the role of macrophage FMRP in atherosclerosis, we generated a myeloid Fmr1-deficient mouse model (myFmr1^{-/-}) and induced hyperlipidemia (FIG. 5F). As expected from the systemic deletion data shown in FIG. 5A, the body weight, plasma glucose and TPC were indistinguishable between the myFmr1^{-/-} and myFmr1^{+/+} genotypes (FIG. 5R-5T). in general, myeloid-specific FMRP-deficiency paralleled the results obtained for Fmr1^{-/-} mice: it resulted in significant reduction in atherosclerotic lesions in en face aorta preparations (FIG. 5G). Myeloid specific FMRP-deficiency did not alter aortic root lesion area but significantly reduced foam cell area (FIG. 5H and 5I). The necrotic core area in the lesions from myFmr1^{-/-} mice was also significantly less than in myFmr1^{-/-} lesions (FIG. 5J), indicating improved AC clearance by Fmr1-macrophages in plaques and supporting our observations that FMRP deficiency increases macrophage efferocytosis in vitro and in vivo (FIG. 3A-3G). Thus, comparing the data obtained with the Fmr1^{-/-} mice with those obtained from the myFmr1^{-/-} mice indicates that the atheroprotective effects observed in Fmr1^{-/-} mice are mostly, if not exclusively, due to FMRP's role in myeloid cells such as macrophages.

To test the notion that IRE1 functions upstream of FMRP, we next investigated the impact of IRE1 kinase inhibition on atherosclerosis. To this end, we fed Apoe^{-/-} mice with WD for 12 weeks and injected them with AMG-18 or vehicle once daily for the last 4 weeks of WD (FIG. 6A). We observed no significant differences in body weight, plasma glucose or TPC, and the number of circulating immune cells between the groups (FIG. 6K-6N). AMG-18 engaged its molecular target effectively in the treatment group (as assessed by reduced IRE1 autophosphorylation) (FIG. 6O). IRE1 kinase inhibition led to a decrease in atherosclerotic lesions in en face aorta preparations (FIG. 6B). As shown above for the Fmr1^{-/-} mice, the aortic root lesion area was not different between the groups (FIG. 6C), but foam cell area as well as necrotic core area were significantly decreased (FIGS. 6D and 6E).

We also assessed the impact of twice daily injections of the same dose of AMG-18 on Apoe^{-/-} mice that were fed with WD for 12 weeks (FIG. 6F). While we noted a decrease in body weight ratio and TPC, there were no significant differences in plasma glucose levels similar to once daily injection (FIG. 6P-6R), and the inhibitor engaged its molecular target effectively (as assessed by reduced IRE1 autophosphorylation) (FIG. 6S, 6T). IRE1 kinase inhibition led to significant decrease in atherosclerotic lesions in en face aorta preparations (FIG. 6G). AMG-18 injection did not alter aortic root lesion area but significantly decreased foam cell area and necrotic core area (FIG. 6H-6J). Collectively, our findings thus demonstrate that the inhibition of IRE1 kinase activity by a small molecule inhibitor or genetic ablation of FMRP, its kinase substrate, in macrophages can reduce the progression of hypercholesterolemia-induced atherosclerosis.

### Discussion

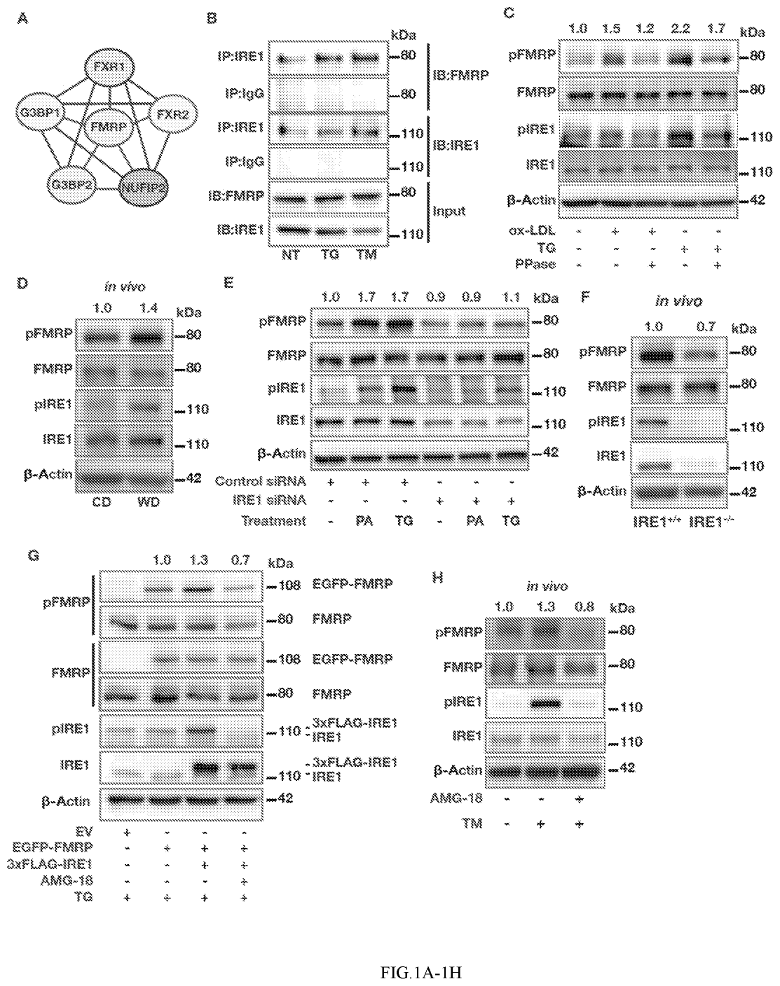

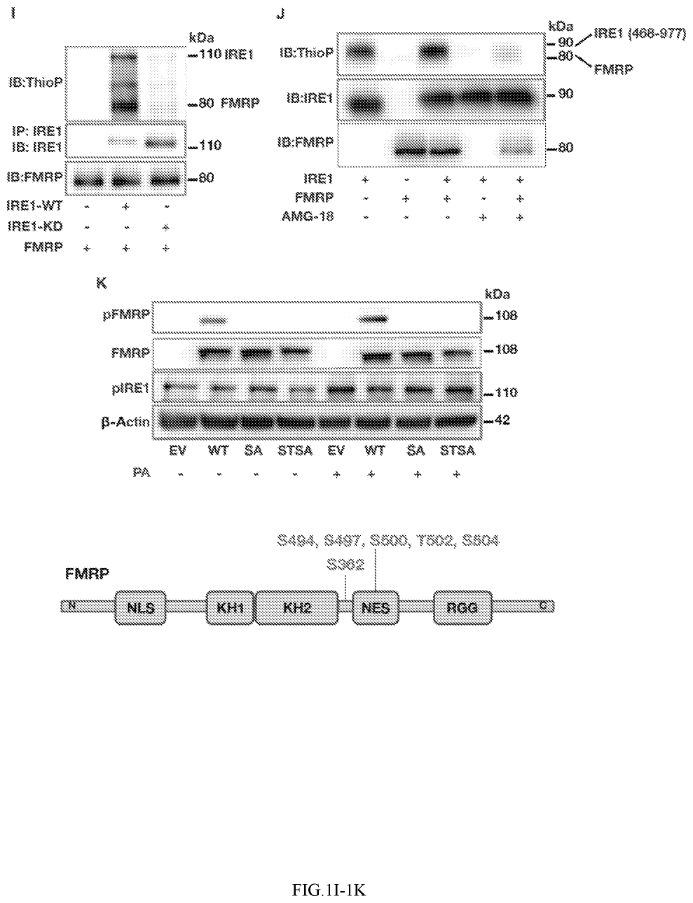

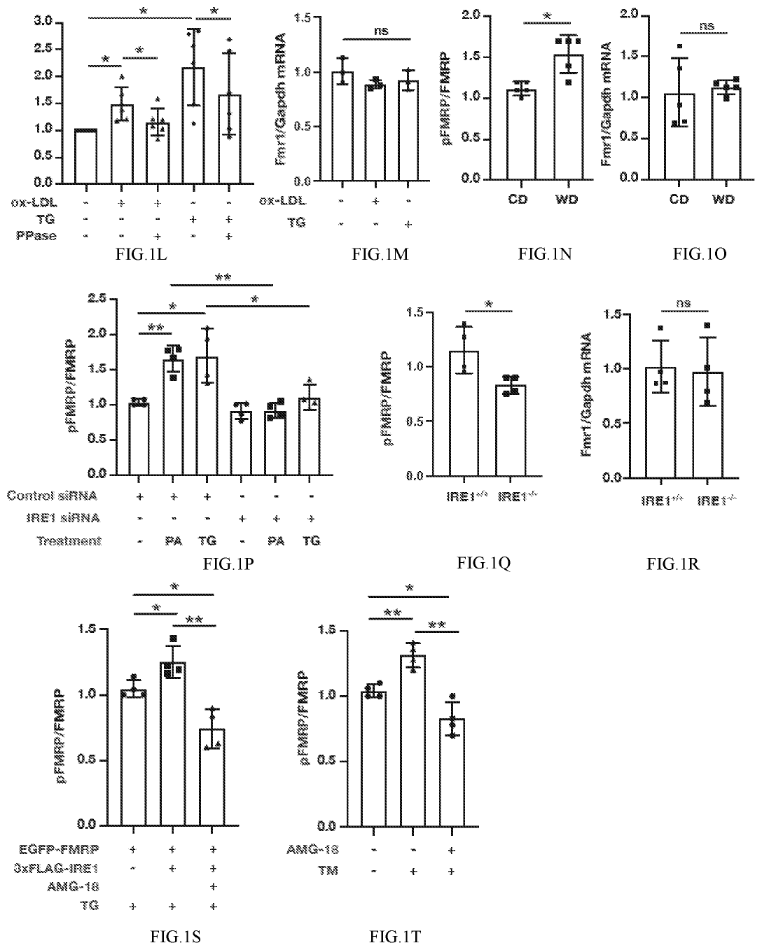

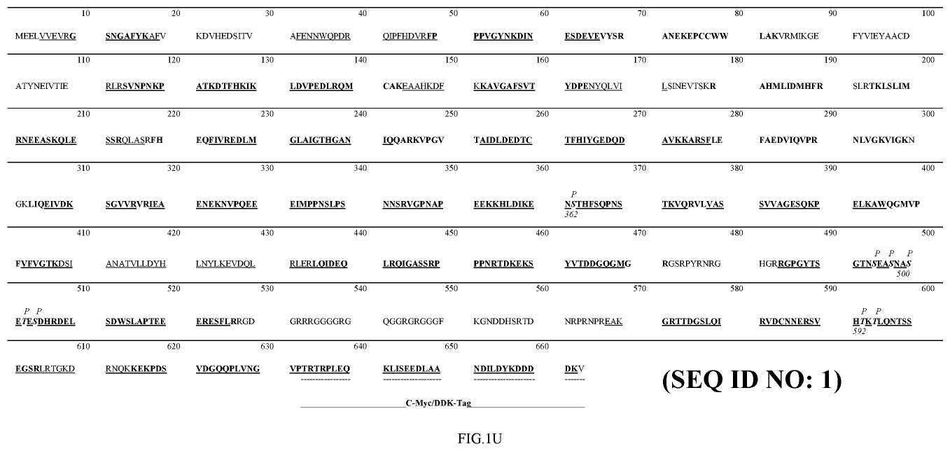

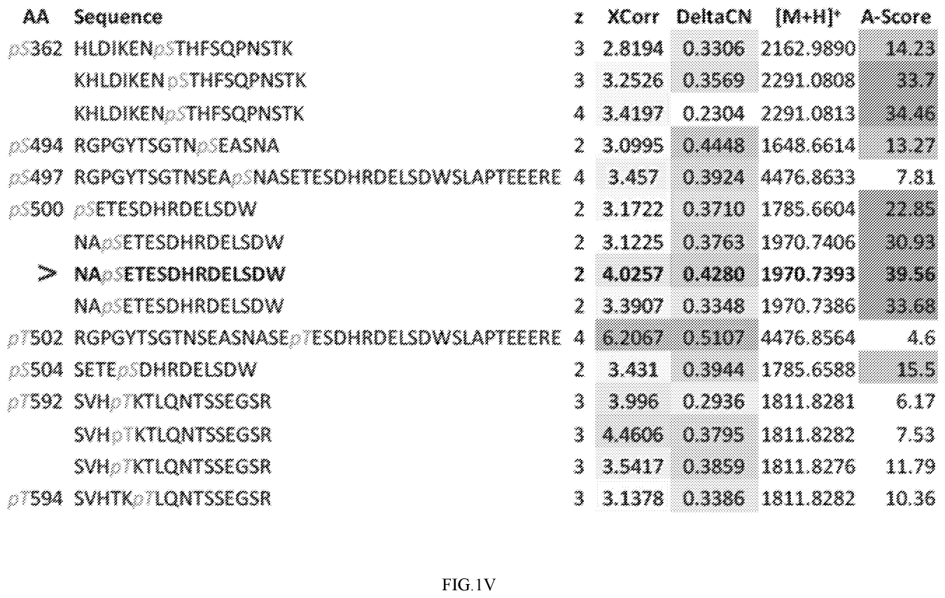

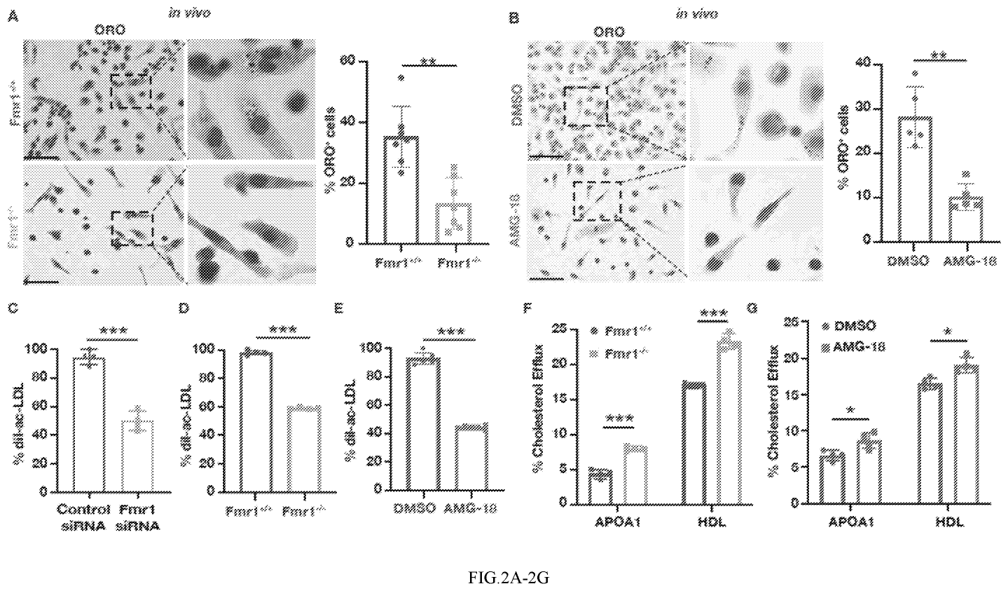

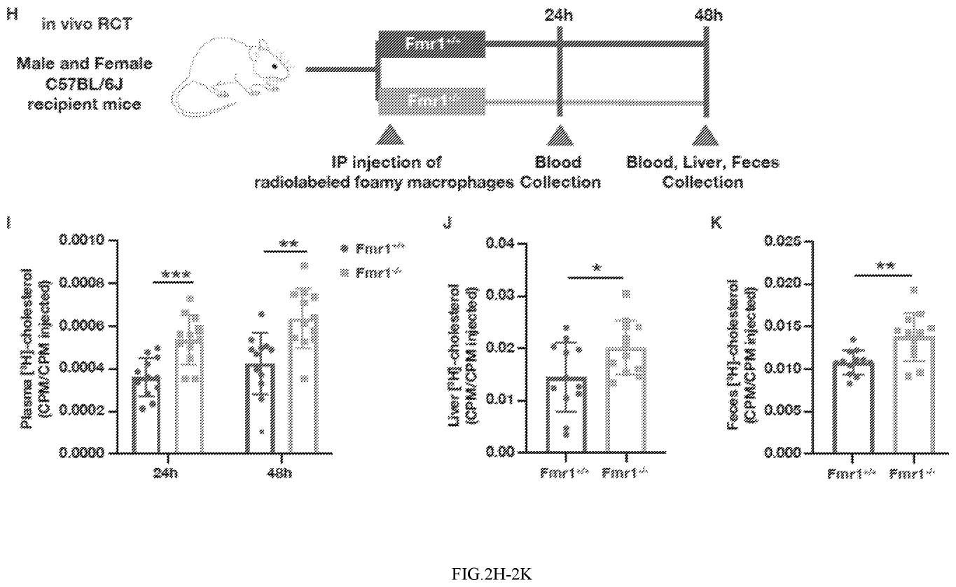

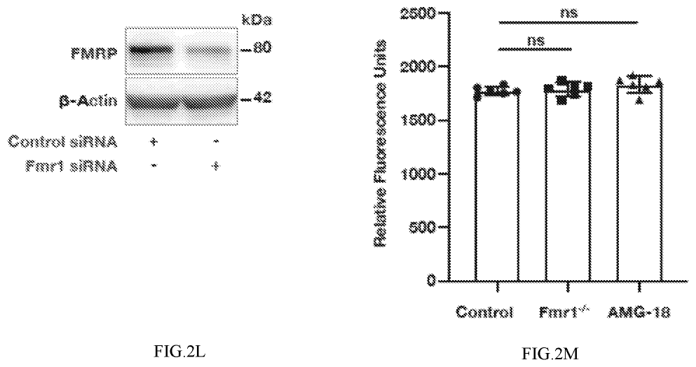

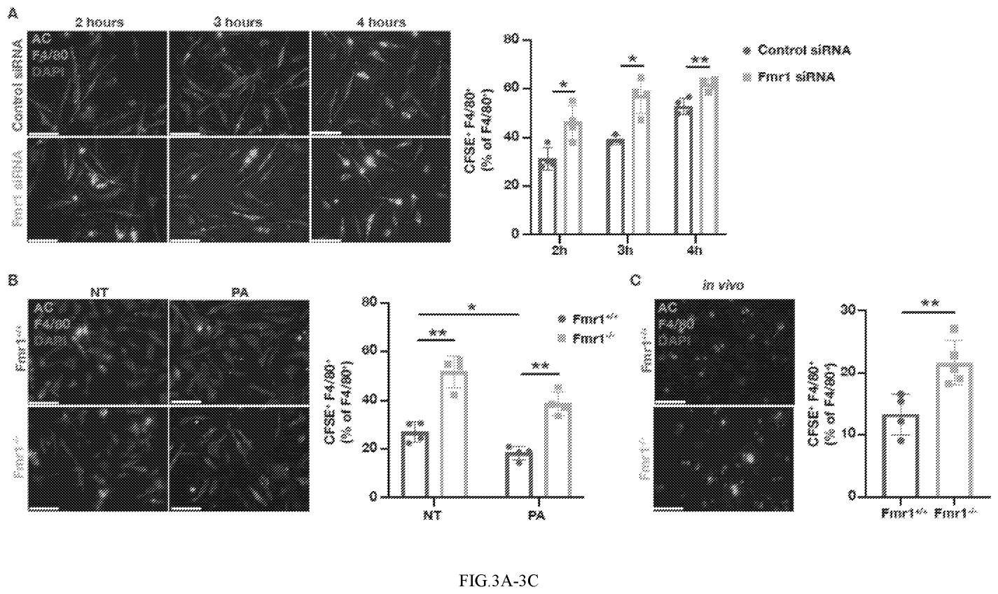

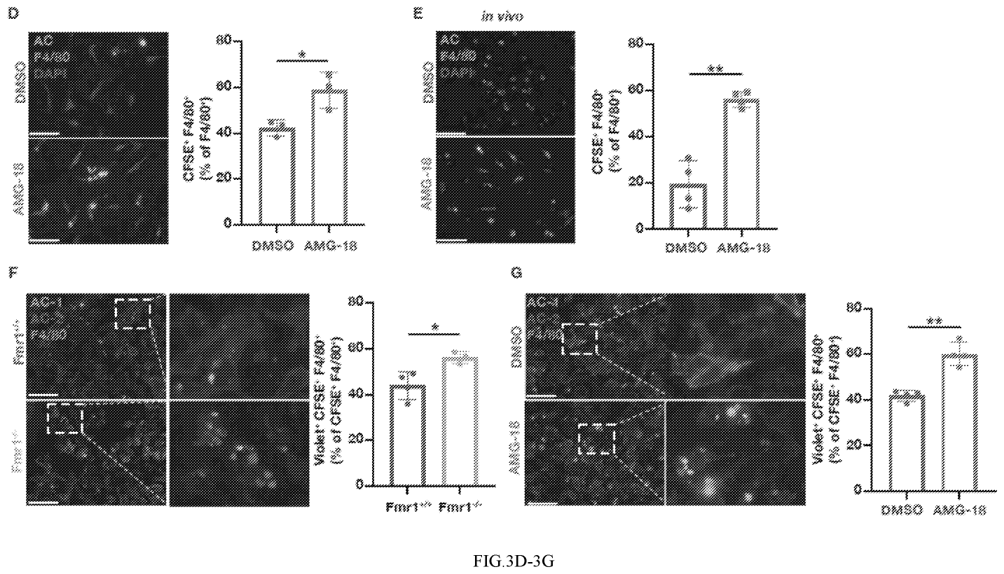

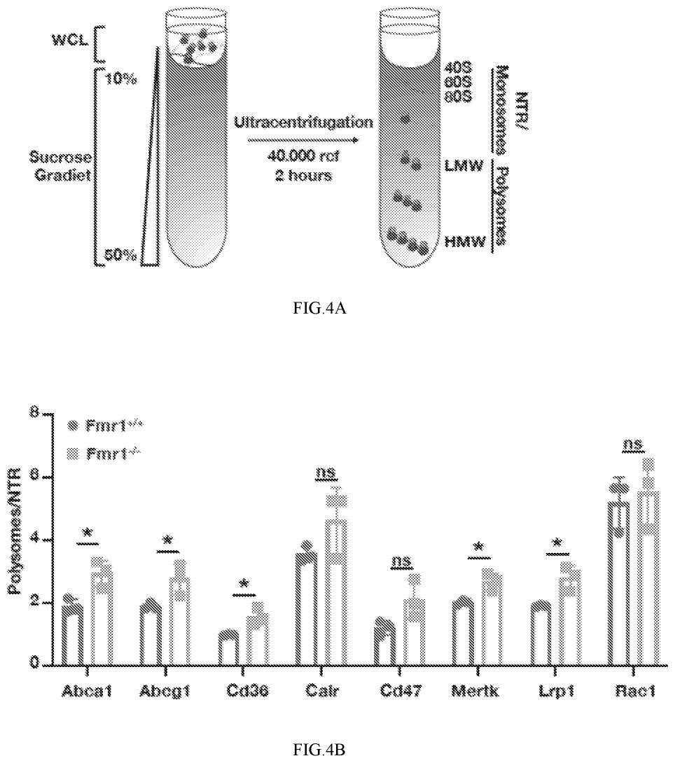

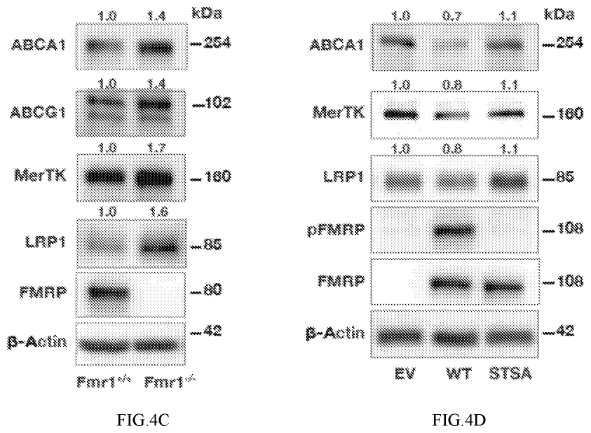

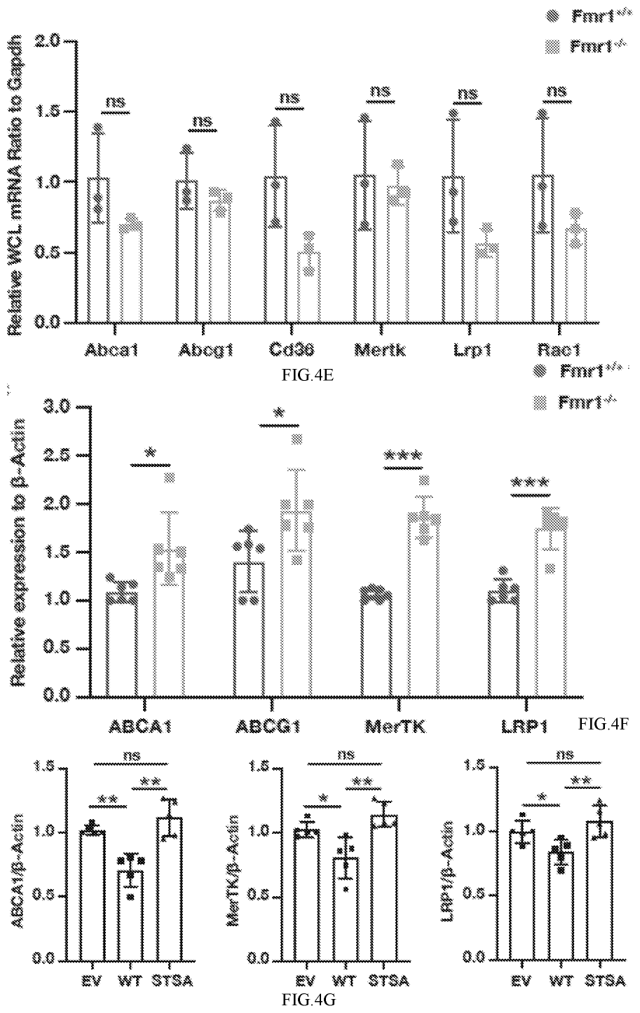

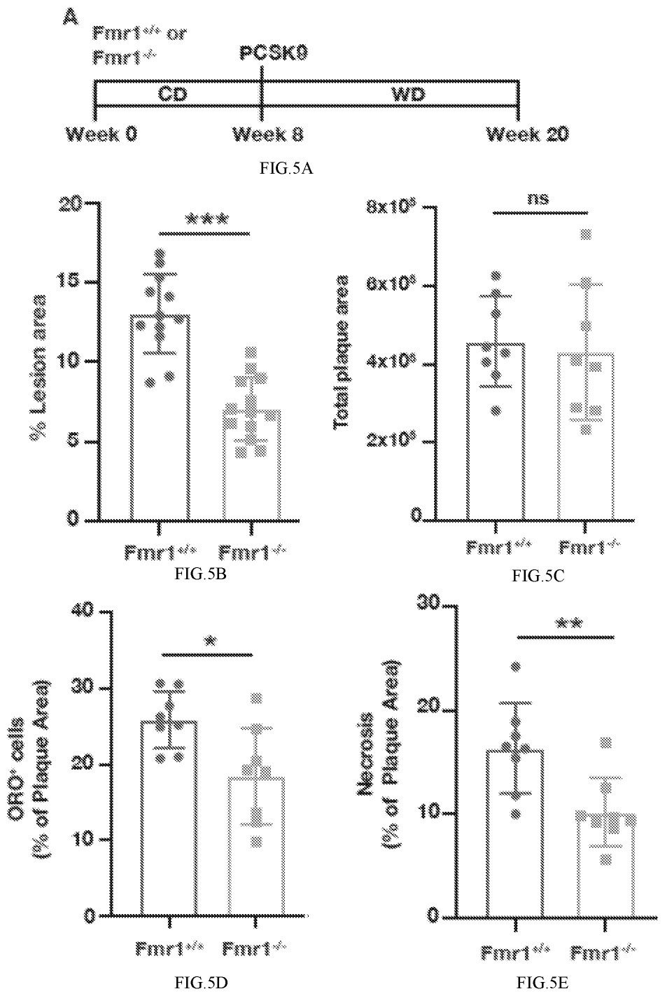

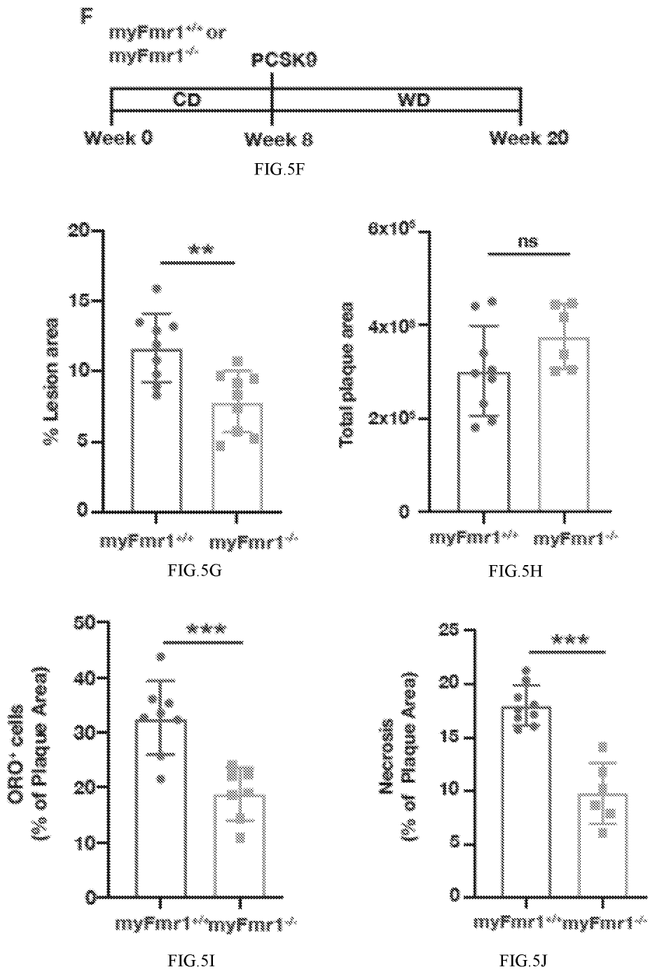

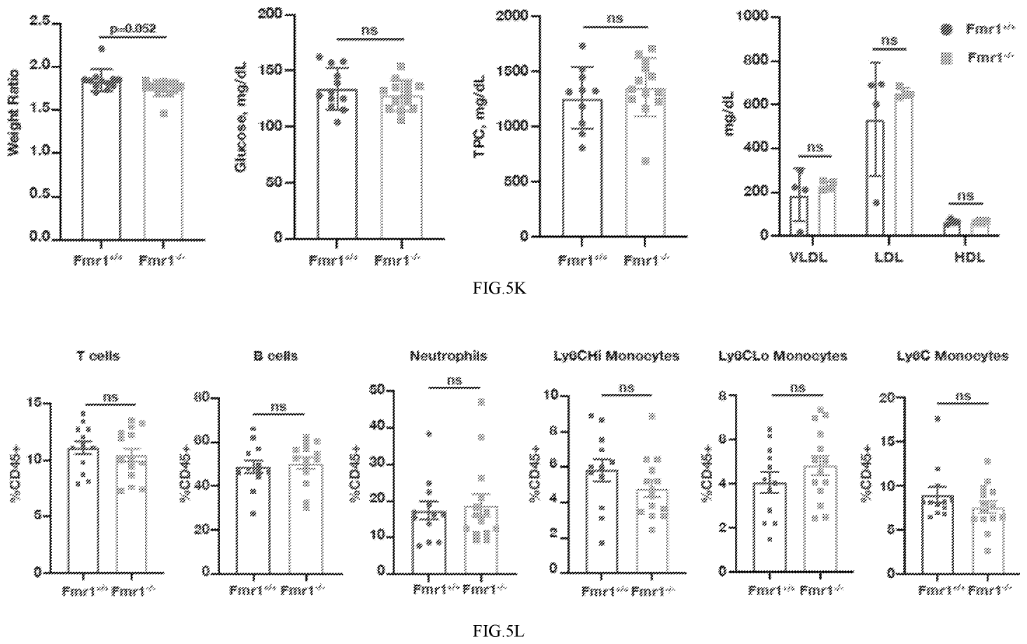

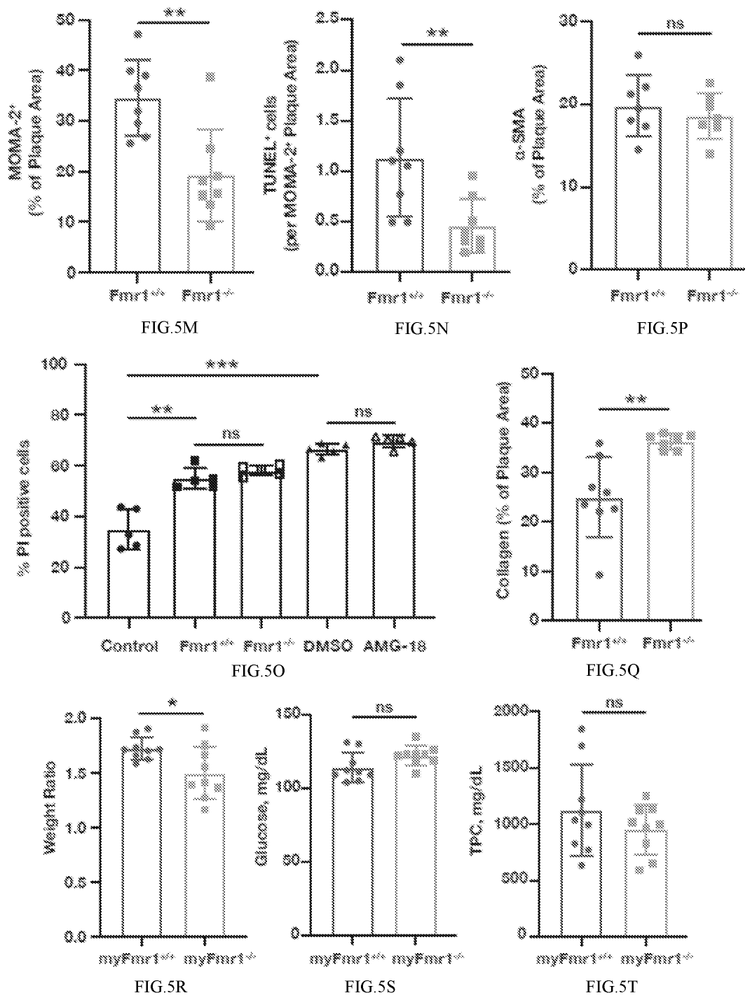

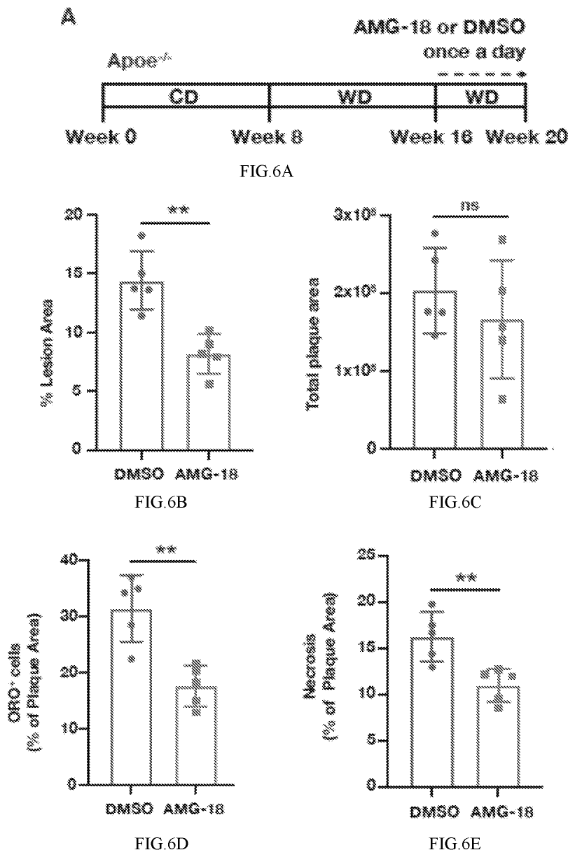

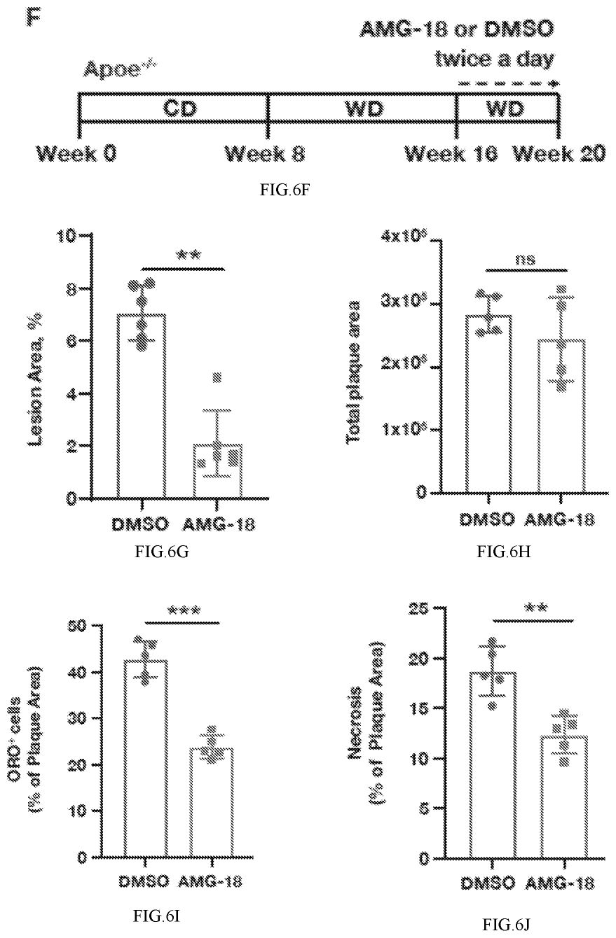

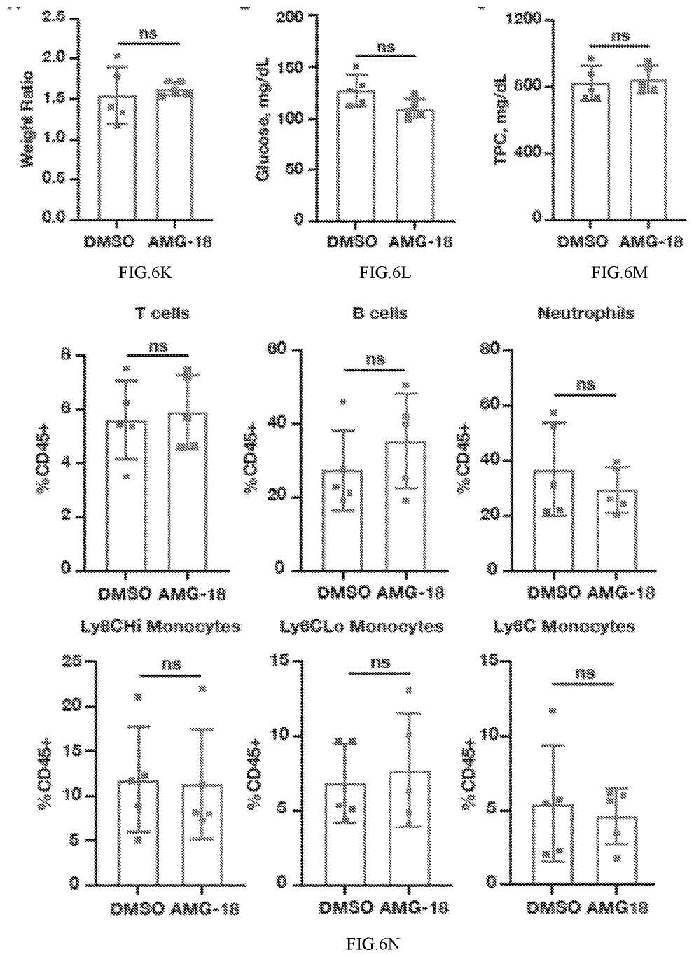

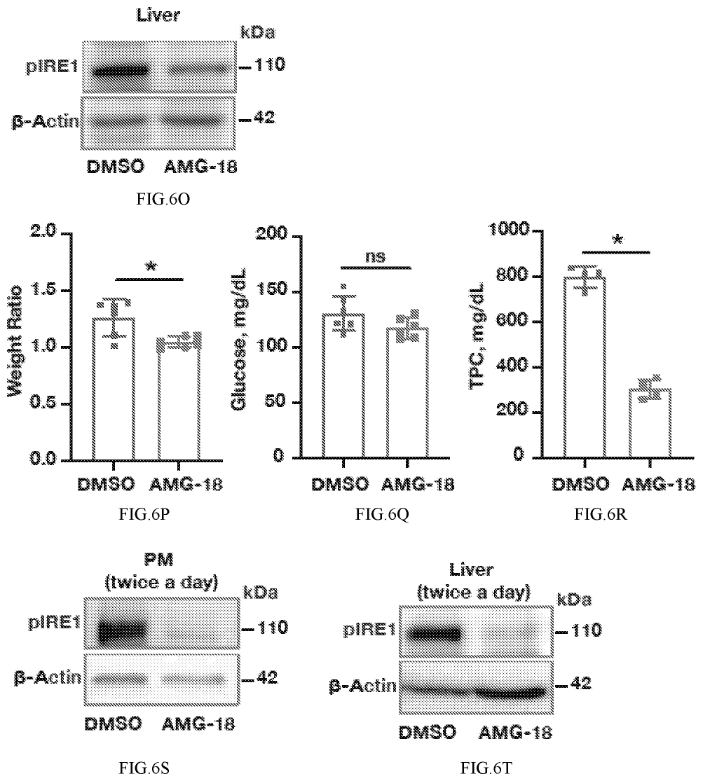

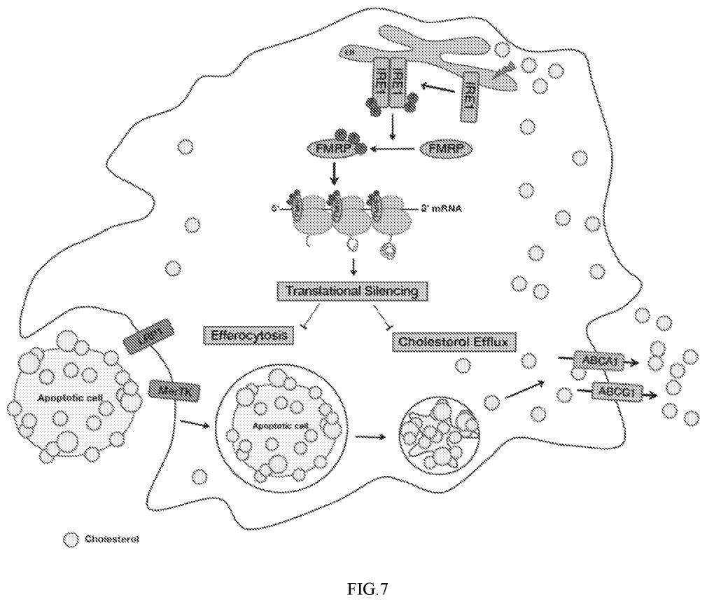

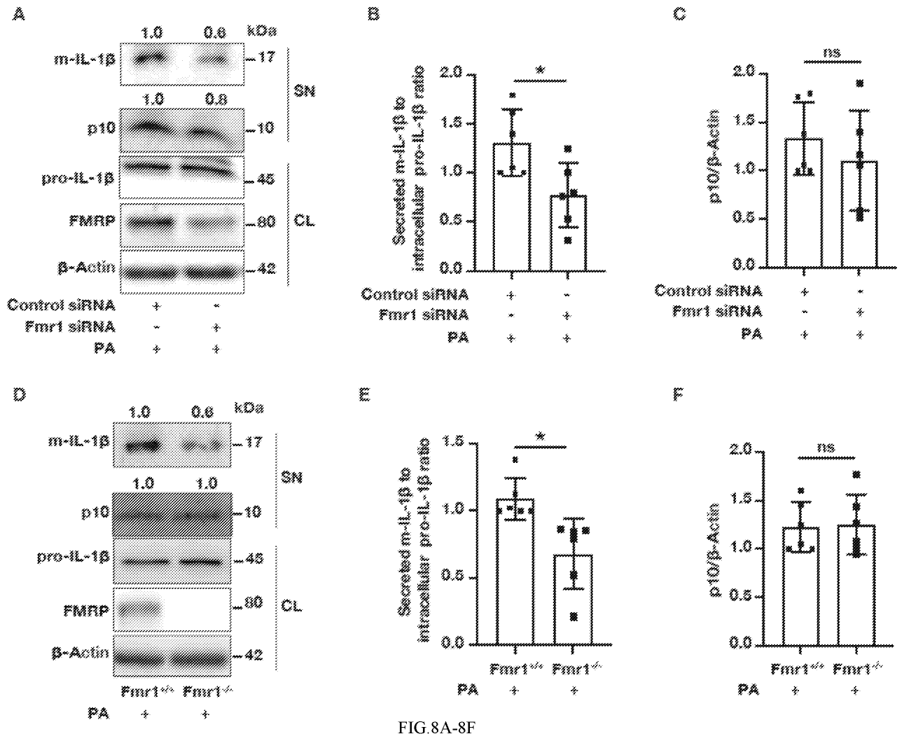

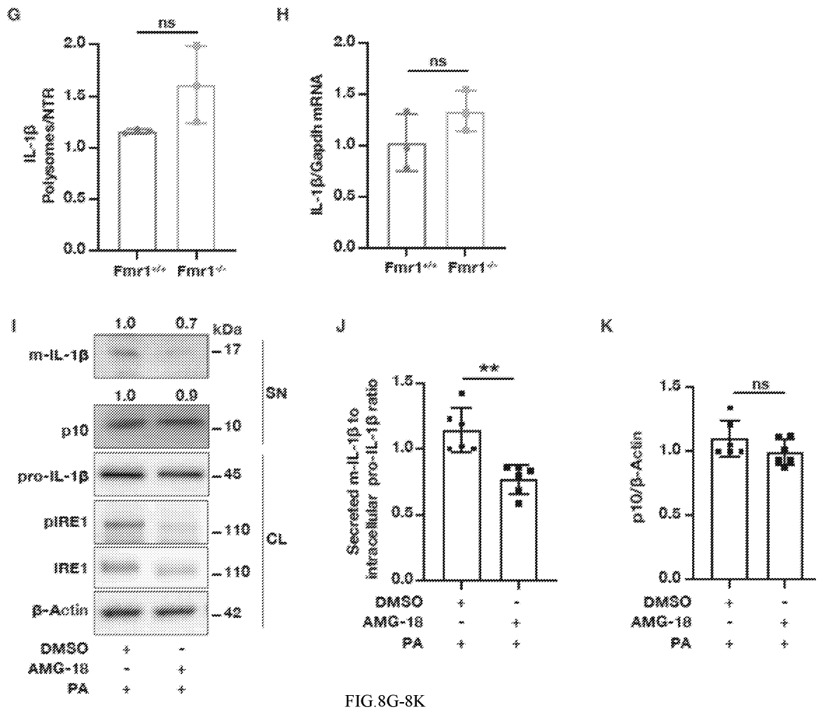

Chronic lipid accumulation in the ER membranes (for example, during obesity and in hyperlipidemia) has been shown to impair ER functions and activate UPR signaling. ER stress is an important primer for sterile inflammation that drives insulin resistance and atherogenesis. Moreover, alleviating ER stress by inhibiting either PERK or IRE1 signaling prevents atherosclerosis progression. But how does chronic IRE1 kinase activation contribute to the atherosclerotic process? Our work revealed that in ER-stressed macrophages phosphorylation of FMRP, an RBP functioning as a translational suppressor, is enhanced in an IRE1 kinase-dependent manner. This phosphorylation event results in a gain-of-function for FMRP, which leads to enhanced translational suppression of cholesterol transporters and efferocytosis receptors. Prior work focused almost exclusively on FMRP's role in neurons in the context of its devastating role in FXS pathology. Our work reveals a new function for FMRP in macrophages. FMRP's contribution to atherosclerosis was not investigated prior to this study, perhaps because Fmr1 lies on the X chromosome and most genome wide association studies in humans focus on variants on autosomal chromosomes excluding sex chromosomes. While characterizing the impact of IRE1 kinase activity and its phosphorylation substrate proposed herein, FMRP, on macrophage functions, our current study revealed a role for an “IRE1-FMRP signaling axis” in the regulation of macrophage cholesterol trafficking and efferocytosis, which are among the primary cellular mechanisms that can regress atherosclerosis (FIG. 7).

To gain insight into the physiological role of macrophage FMRP, we investigated the consequences of FMRP-deficiency on macrophage biology that are relevant to atherosclerosis development and plaque regression, such as foam cell formation, cholesterol efflux and efferocytosis. Our findings show that FMRP has a prominent role in all three processes. Additionally, our observations are consistent with the finding that several mRNAs encoding cholesterol transporters and efferocytosis receptors interact with FMRP in prior RNA crosslinking-immunoprecipitation (CLIP) experiments. Our data show that the absence of FMRP in macrophages increases the occupancy of these mRNAs in the polysome fraction, paralleling the increased expression of their corresponding protein products. In agreement with our observations in cells, FMRP-deficiency in macrophages increased RCT and efferocytosis in mice. Moreover, systemwide and myeloid-specific Fmr1 knock-out mice were protected from hypercholesterolemia-induced atherosclerosis progression. In agreement with this notion, the IRE1 kinase-specific inhibitor, AMG-18, phenocopies the beneficial impact of FMRP deficiency (such as enhanced cholesterol efflux and efferocytosis) in macrophages. The impairment of RCT and efferocytosis by IRE1-FMRP signaling implies that interventions to ablate FMRP in a macrophage-specific manner and in the adult organism could promote atherosclerotic plaque stabilization and hinder plaque progression, while escaping adverse impact of FMRP-deficiency on neuronal development.

We did not find Fmr1-deficiency associated changes in plasma cholesterol or lipoprotein levels in our atherosclerosis mouse models, perhaps due to the severe hypercholesterolemia induced by both genetic and dietary interventions.

Small molecule inhibitors that are specific for IRE1's RNase activity (e.g., STF-083010 and 4μ8C) prevented lipid-induced inflammasome activation and secretion of mature interleukin-1β (m-IL-1β) and m-IL-18 in both mouse and human macrophages while reducing hyperlipidemia-induced m-IL-1β and m-IL-18 production and atherosclerotic plaque size in mice. Herein, FMRP suppression also reduces m-IL-1β secreted from macrophages but without altering inflammasome activation (FIG. 8A-8F). Knocking out FMRP from macrophages has no effect on IL-1β mRNA levels in the whole cell lysate or in the polysomes, indicating against transcriptional or translational control over these cytokines' production (FIGS. 8G and 8H). As expected, inhibition of IRE1 kinase also reduces m-IL-1β (FIG. 8I-8K). Since the inhibition of IRE1 kinase-FMRP axis leads to a marked upregulation of cholesterol efflux in macrophages (as shown in our study), it is plausible that increased demand for ABCA1 for cholesterol efflux could prevent m-IL-1β secretion through this route.

Without being bound to a particular theory, it is plausible that during ER stress, IRE1-induced FMRP phosphorylation, mediated by S500 phosphorylation and then spreading to adjacent serines and threonines, enhances phase-separation of FMRP and leads to the redistribution of FMRP-bound RNA into cytoplasmic granules, where they will not be translated. This would, in effect, reduce the protein folding stress and improve ER homeostasis. Indeed, ER stress induces stress granule formation, which contain mRNAs, RBPs (including FMRP), 40S ribosomal subunits and translational pre-initiation factors.

Finally, our findings demonstrate that IRE1 kinase-specific inhibitor, AMG-18, can phenocopy the beneficial impact of FMRP deficiency (such as enhanced cholesterol efflux and efferocytosis) in macrophages. Our data strongly supports that this new IRE1 kinase target-FMRP's expression in macrophages plays an important role in cholesterol efflux and apoptotic cell clearance and is an important contributor to atheroprotection offered by IRE1 kinase inhibition. Our study provides mechanistic insight into the translational regulation of cholesterol efflux and efferocytosis by macrophages during ER stress, highlighting IRE1 and its effector FMRP as a promising molecular target for regulating atherosclerosis. The small molecule inhibitors of IRE1 kinase activity represent a translational opportunity in combating atherosclerosis.

### Techniques and Materials

General study design: Three or more independent replicates were performed for cell-based experiments. Mice were randomly assigned to independent cohorts, and data analysis was performed blind. The only elimination criteria used for mouse studies was based on health and as advised by a veterinarian.

Reagents and plasmids: WT-Ire1-pcDNA3, and Kinase-dead IRE1 mutant (K599A)-pcDNA3 plasmids were generated in Dr. Peter Walter's laboratory (University of California, San Francisco). Fatty acid free bovine serum albumin was from Gold Biotechnology (A-421-250). Lipofectamine 3000 (L3000001) and EDTA (15575020) were purchased from Invitrogen. Polyethylemine (PEI; molecular weight 25,000) was from Polysciences. Neon transfection system kit (MPK10096) was from Thermo Scientific and used with the Neon electroporation system, also from Thermo Scientific. L-Glutamine, Dulbecco's modified Eagle's Medium (DMEM), penicillin/streptomycin (P/S), fetal bovine serum (FBS), Roswell Park Memorial Institute (RPMI)-1640 medium, phosphate buffer saline (PBS), CELLTRACE™ CFSE cell proliferation kit (C34554), CELLTRACE™ Violet Cell Proliferation Kit (C34557), Fmr1 siRNA (AM16708), Control siRNA (negative control, AM4611), Revert Aid First strand cDNA synthesis kit (K1691) and Formaldehyde (31901) were from Thermo Scientific. Protease inhibitor cocktail (P8340), phosphatase inhibitor cocktail-3 (P0044), Dimethyl sulfoxide (DMSO, D8418), Trypsin, Ampicillin, palmitic acid (PA; P0500), Oil Red O solution (01391), Hematoxylin (HHS32), Lipoprotein-deficient serum (S5519), CC mount (C9368), ECL™ Prime Western Blotting Detection Reagent (GERPN2232), fatty-acid free BSA (A6003), Sucrose (S0389) and thioglycolate solution (70157) were purchased from Sigma. Dil-labeled ac-LDL (770201-7) was from Kalen Biomedical. The IRE1 Kinase inhibitor, AMG-18 were from Tocris. Primary antibodies used for immunoblotting were from the following companies: anti-pIRE1 (phsopho-S724; 124945), anti-FMRP (ab17722), anti-LRP1 (ab92544), anti-α-SMA (ab5694), Anti-IL-1 beta antibody (ab9722), Anti-pro Caspase1+p10+p12 antibody [EPR16883] (ab179515) and anti-Thiophosphate ester antibody (ab133473) antibodies purchased from Abcam; anti-pFMRP (phosphor-S499; p1125-499) from PhosphoSolutions; anti-IRE1 (3294), anti-LRP1 (64099) and anti-FMRP (4317) from Cell-Signaling; anti-FMRP (NBP02-01770), anti-ABCA1 (NB400-105) and anti-ABCG1 (NB400-132) from Novus Biologicals; anti-β-Actin-horse radish peroxidase (47778) and Secondary IgG-Goat (sc-2354) from Santa Cruz Biotechnology, anti-MOMA-2 from Bio-Rad, anti-PE-F4/80 (123110); anti-FMRP (834601), Propidium Iodide Solution, PI (421301), Cell Staining Buffer (420201) and TruStain FcX (anti-CD16/32, 101319) from Biolegend. Active ERN1 human recombinant protein (E31-11G) was from Biotech. FMRP human recombinant protein (TP322699) was from Origene. Cholesterol Uptake Assay Kit (ab236212), cholesterol efflux assay kit (ab 196985), Masson Trichrome (04-010802), Fluoroshield mounting reagent with DAPI (ab104139), ATP-γ-S (ab138911) and p-Nitrobenzyl mesylate (PNBM, ab138910) were purchased from Abcam. Aqua-Mount (41799-008) was from VWR. Cremophor EL (238470) and Cycloheximide (239763) were purchased from EMD Millipore. TUNEL (In situ Cell Death detection Kit, Fluorescenin; 11684795910) were purchased from Roshe. Hematoxylin and Eosin Stain Kit (H-3502) were purchased from Vector Laboratories. Wako Diagnostics total cholesterol E kit (NC9138103) were purchased from Fujifilm Medical Systems U.S. Pat. No. 99,902,601. TRIsure (BIO-38033) was purchased from Bioline. Power-Up-SYBR green (A25742) was purchased from Applied Biosystems. 3H-cholesterol (NET139001MC) were purchased from Perkin Elmer. HBSS (14175095) was purchased from Gibco. Anti-MERTK (AF-591-SP) antibody was purchased from R&D. Secondary IgG-Rabbit (5220-0337) and Secondary-IgG-Mouse (5220-0341) were purchased from SeraCare. Lambda phosphatase enzyme (sc-200312) was purchased from Santu Cruz.

Mice studies and treatments: C57BL/6 (WT, Fmr1^{+/+}), Fmr1^{-/-} and Apolipoprotein E-deficient (Apoe^{-/-}) mice were purchased from Jackson Laboratory. IRE1 conditional knock out (Ire1α^{flox/flox}) mice were a kind gift from Dr. Takao Iwawaki (Kanazawa Medical University, Japan) and characterized before (Iwawaki et al, 2009). Ire1α^{flox/flox} mice were inter-crossed from with LysM^{cre} mice purchased from Jackson Lab (004781) to obtain Ire1α^{flox/flox}, LysM^{cre} mice (IRE1^{-/-}), which had a myeloid-specific Ire1α gene deletion. Fmr1 conditional knock out (Fmr1^{flox/flox}) mice were a kind gift from dr. David Nelson. Fmr1^{flox/flox} mice were inter-crossed with LysM^{cre} mice purchased from Jackson Lab (004781) to obtain myeloid Fmr1-deficient (myFmr1^{-/-}) mice. Starting at 8 weeks of age, Fmr1^{-/-}, Fmr1^{-/-}, myFmr1/or myFmr1^{-/-} mice were injected with 1×10¹⁰ AAV_PCSK9 (AAV8-D377Y-mPCSK9, Vector BiolabsAAV-268246) via tail vein, then fed with normal chow or high cholesterol/high fat atherosclerotic mouse diet from Envigo (TD.88137) for 6 to 16 weeks. Apoe^{-/-} mice were fed with WD (12 weeks) and intraperitoneally injected with vehicle (DMSO) or AMG-18 (30 mg/kg) once or twice a day in the last 4 weeks of WD. C57BL/6 were injected with Tunicamycin (TM, 1 mg/kg) and with AMG-18 (30 mg/kg) or vehicle (DMSO) in 20% vol/vol Cremophor EL saline solution. 8 hours later peritoneal macrophages were isolated by thioglycolate elicitation for further analysis. Mice were kept under specific pathogen-free conditions with food and water ad libititum. Both female and male mice were used for experiments. All animal experiments were performed according to protocols approved by the Experimental Animal Ethical Care Committees at Bilkent University, Ankara, Turkey or Cedars Sinai Medical Center, Los Angeles, USA or the University of Ottawa Animal Care Committee, Ottawa, ON KIN 6N5, Canada.

Isolation of Peritoneal macrophages: 3% thioglycolate solution were injected to mice intraperitoneally and peritoneal macrophages were collected 4 days after by washing the peritoneal cavity with ice-cold PBS (10 ml). Cells were centrifuged at 500×g for 5 min at 4° C. and resuspended in RPMI medium in cell culture plates. Macrophages were incubated at 5% carbon dioxide incubator at 37° C. for 30 minutes to attach and non-adherent cells were removed along with media. Cells were rinsed with PBS and used for protein isolation or RNA isolation.

Isolation of Bone Marrow-Derived Macrophages (BMDM): Bone marrows were collected form the tibia and femurs of mice into RPMI containing 1% Penicillin/streptomycin (P/S) cocktail. After filtering through a cell strainer (BD, 352350), cells were centrifuged at 500×g for 5 minutes and re-suspended in RPMI enriched with 20% L929 cells conditioned medium, 10% heat-inactivated fetal bovine serum (FBS) and 1% P/S cocktail, followed by growth on petri dishes and differentiation to macrophages for 5-10 days.

Cell lines: Fmr1^{-/-} mouse embryonic fibroblasts (MEF) were generated in Dr. David Nelson's laboratory (Baylor College of Medicine, Houston, Texas). HEK293T, Jurkat (human T lymphocytes) and L-929 (mouse fibroblasts) cells were obtained from ATCC. Cells were cultured in RPMI or DMEM supplemented with 10% heat-inactivated FBS and % 1 P/S cocktail. Cells were cultured in a humidified CO2 incubator at 37° C. All cells were regularly tested for mycoplasma contamination

Transfection: 60-80% confluent HEK293T, WT or Fmr1^{-/-} MEF cells were transfected using Lipofectamine 3000 or Polyethylemine (PEI). BMDM and HEK293T cells were electroporated either with IRE1-, Fmr1-(100 nM) or control-siRNA using Neon electroporator (Thermo Scientific) as per specific conditions provided by the manufacturer. 24-36 hours after transfection, cells were treated with PA or TG to induce ER stress.

Palmitate (PA)/bovine serum albumin (BSA) complex preparation: PA was dissolved in absolute ethanol to yield a stock concentration of 500 mM and stored at −80° C. Stock PA was diluted to working concentration and suspended with % 1 fatty acid free-BSA in serum free RPMI growth medium by mixing at 55° C. for 15 minutes.

Western blot analysis: Cells were lysed in lysis buffer (50 mM HEPES pH: 7, 9, 100 mM NaCl, 10 mM EDTA, 10 mM NaF, 4 mM NaPP, 1% Triton, 1 mM phenylmethanesulfonylfluoride (PMSF), 1× phosphatase inhibitor cocktail 3 and 1× (10 μM) protease inhibitor cocktail. After centrifugation, clear lysates were mixed with sodium dodecyl sulfate (SDS) loading dye and heated at 95° C. for 5 minutes before loading on SDS-polyacrylamide gel (SDS-PAGE) gels. After separation according to protein molecular weights on these gels, samples were transferred to polyvinylidene difluoride (PVDF) membrane. Blocking and antibody incubation of the membranes were carried out in tris-buffered saline (TBS) buffer prepared with 0.1% Tween-20 (v/v) and 5% (w/v) dry milk or BSA. ECL prime reagent were used to develop the membranes and images were captured with ChemiDoc (BioRad). Antibody dilutions: anti-pIRE1 1:2000, anti-FMRP 1:2000, anti-LRP1 1:5000, anti-IL-1 beta 1:500, Anti-pro Caspase1+p10+p12 1:2000, anti-Thiophosphate ester 1:5000, anti-pFMRP (phosphor-S499) 1:2000, anti-IRE1 1:2000, anti-ABCA1 1:1000, anti-ABCG1 1:1000, Anti-MERTK 1:1000, anti-β-Actin-horse radish peroxidase 1:5000, Secondary IgG-Goat 1:10000, Secondary IgG-Rabbit 1:10000, Secondary-IgG-Mouse 1:10000.

RNA Isolation and Quantitative Reverse Transcription Polymerase Chain Reaction (qRT-PCR): Total RNA was isolated using TRIsure. RNA extractions were than reverse transcribed by using Revert Aid First strand cDNA synthesis kit to complementary deoxyribonucleic acid (cDNA) according to manufacturer's protocol. Using specific primers, cDNAs were amplified on Rotor Gene (Qiagen). Power-Up-SYBR green (Applied Biosystems, A25742) was used for qRT-PCR reaction. The following PCR primers were used for mRNA expression analysis:

| | mmu-Fmr1-F |
|---|---|
| (SEQ ID NO: 22) | |
| | 5′ CCGAACAGATAATCGTCCACG 3′ |
| | mmu-Fmr1-R |
| (SEQ ID NO: 23) | |
| | 5′ ACGCTGTCTGGCTTTTCCTTC 3′ |
| | mmu-Abca1-F |
| (SEQ ID NO: 24) | |
| | 5′ AAAACCGCAGACATCCTTCAG 3′ |
| | mmu-Abca1-R |
| (SEQ ID NO: 25) | |
| | 5′ CATACCGAAACTCGTTCACCC 3′ |
| | mmu-Abcg1-F |
| (SEQ ID NO: 26) | |
| | 5′ GGTCCTGACACATCTGCGAA 3′ |
| | mmu-Abcg1-R |
| (SEQ ID NO: 27) | |
| | 5′ CAGGACCTTCTTGGCTTCGT 3′ |
| | mmu-Mertk-F |
| (SEQ ID NO: 28) | |
| | 5′ CAGGGCCTTTACCAGGGAGA 3′ |
| | mmu-Mertk-R |
| (SEQ ID NO: 29) | |
| | 5′ TGTGTGCTGGATGTGATCTTC 3′ |
| | mmu-Lrp1-F |
| (SEQ ID NO: 30) | |
| | 5′ GCCTACACCTGGAGAGATAGC 3′ |
| | mmu-Lrp1-R |
| (SEQ ID NO: 31) | |
| | 5′ GGCAACTTACGAGCAGGCT 3′ |
| | mmu-Cd36-F |
| (SEQ ID NO: 32) | |
| | 5′ GTGCTCTCCCTTGATTCTGC 3′ |
| | mmu-Cd36-R |
| (SEQ ID NO: 33) | |
| | 5′ CTGCACCAATAACAGCTCCA 3′ |
| | mmu-Cd47-F |
| (SEQ ID NO: 34) | |
| | 5′ TGGTGGGAAACTACACTTGCG 3′ |
| | mmu-Cd47-R |
| (SEQ ID NO: 35) | |
| | 5′ CGTGCGGTTTTTCAGCTCTAT 3′ |
| | mmu-Calr-F |
| (SEQ ID NO: 36) | |
| | 5′ GCAGACCCTGCCATCTATTTC 3′ |
| | mmu-Calr-R |
| (SEQ ID NO: 37) | |
| | 5′ TCGGACTTATGTTTGGATTCGAC 3′ |
| | mmu-Rac1-F |
| (SEQ ID NO: 38) | |
| | 5′ ATGCAGGCCATCAAGTGTG 3′ |
| | mmu-Rac1-R |
| (SEQ ID NO: 39) | |
| | 5′ TAGGAGAGGGGACGCAATCT 3′ |
| | mmu-IL-1β-F |
| (SEQ ID NO: 40) | |
| | 5′ CAACCAACAAGTGATATTCTCCATG 3′ |
| | mmu-IL-1β-R |
| (SEQ ID NO: 41) | |
| | 5′ GATCCACACTCTCCAGCTGCA 3′ |
| | mmu-Gapdh-F |
| (SEQ ID NO: 42) | |
| | 5′ ATTCAACGGCACAGTCAAGG 3′ |
| | mmu-Gapdh-R |
| (SEQ ID NO: 43) | |
| | 5′ TGGATGCAGGGATGATGTTC 3′ |

The following primers were used to introduce site directed mutagenesis on FMRP plasmids:

| | S500A-F |
|---|---|
| (SEQ ID NO: 44) | |
| | 5′ GCATCAAATGCTGCTGAAGCAGAAGCTGACCACAGAGAC 3′ |
| | S500A-R- |
| (SEQ ID NO: 45) | |
| | 5′ GTCTCTGTGGTCAGCTTCTGCTTCAGCAGCATTTGATGC 3′ |
| | S500-T502-S504A-F |
| (SEQ ID NO: 46) | |
| | 5′ GCATCAAATGCTGCTGAAGCAGAAGCTGACCACAGAGAC 3′ |
| | S500-T502-S504A-R |
| (SEQ ID NO: 47) | |
| | 5′ GTCTCTGTGGTCAGCTTCTGCTTCAGCAGCATTTGATGC 3′ |

Identifying phosphorylation sites on hFMRP using Mass spectrometry: Two in vitro kinase reactions of hFMRP and ERN1, worth 4.5 μg protein each were methanol-chloroform precipitated. Dried pellets were dissolved in either [1] 8 M urea/100 mM triethylammonium bicarbonate (TEAB, Thermo Scientific 90114), pH 8.5, or [2] 100 mM ammonium acetate (Sigma-Aldrich A1542), with or without 8 M urea. Proteins were reduced with 5 mM tris(2-carboxyethyl) phosphine hydrochloride (TCEP-HCl, Thermo Scientific C4709) and alkylated with 10 mM 2-chloroacetamide (Sigma-Aldrich 22790). Proteins dissolved in urea/TEAB were digested at 37° C. in 0.8 M urea/100 mM TEAB, pH 8.5, sequentially with 500 ng Trypsin (Promega V5117) for 17 hours, followed by 500 ng Endoproteinase GluC (NEB P8100S) for 4.5 h and quenched with formic acid, 5% final concentration, while proteins dissolved in urea/TEAB or urea/ammonium acetate were digested with 200 ng Proteinase K (Sigma-Aldrich P2308) at 37° C. for 30 min and heat-quenched at 90° C. for 15 min (similar reactions in ammonium acetate without urea were performed for 30 min or 15 min followed by 16 h digestion with trypsin) ((Baboo et al, 2021). The digest was injected directly onto a 20 cm, 100 μm ID column packed with BEH 1.7 μm C18 resin (Waters 186005225). Samples were separated at a flow rate of 400 nl/min on an nLC 1000 (Thermo LC120). Buffer A and B were 0.1% formic acid in 5% acetonitrile and 0.1% formic acid in 80% acetonitrile, respectively. A gradient of 1-25% B over 110 min, an increase to 40% B over next 20 min, an increase to 90% B over another 10 min and a hold at 90% B for the final 10 min was used for a total run time of 140 min. The column was re-equilibrated with 20 μl of buffer A prior to the injection of sample. Peptides were eluted directly from the tip of the column and nano-sprayed into the mass spectrometer by application of 2.8 kV voltage at back of the column. The Orbitrap Fusion Lumos (Thermo) was operated in data dependent mode. Full MS1 scans were collected in the Orbitrap at 120K resolution with a mass range of 400 to 1500 m/z and an AGC target of 4e5. The cycle time was set to 3 s, and within these 3 s, the most abundant ions per scan were selected for CID MS/MS in the ion trap with an AGC target of 2e4 and minimum intensity of 5000. Maximum fill times were set to 50 ms and 35 ms for MS and MS/MS scans, respectively. Quadrupole isolation at 1.6 m/z was used, monoisotopic precursor selection was enabled, charge states of 2-7 were selected, and dynamic exclusion was used with an exclusion duration of 5 s. Samples were also analyzed with HCD fragmentation (35 NCE) and detection at 7500 resolution.

Protein and peptide identification were done with Integrated Proteomics Pipeline-IP2 (Integrated Proteomics Applications). Tandem mass spectra were extracted from raw files using RawConverter and searched with ProLuCID against a concatenated database comprising of amino acid sequences from vendors for FMRP, hERN1 and Endoproteinase GluC, UniProt reference proteome of Escherichia coli K12 (UP000000625) Homo sapiens (UP000005640). The search space included all fully-tryptic and half-tryptic peptide candidates (no enzyme specificity for sample treated with Proteinase K). Carbamidomethylation (+57.02146) was considered a static modification on cysteine, and phosphorylation (+79.966331) was considered a differential modification on serine/threonine/tyrosine. Data was searched with 50 ppm precursor ion tolerance and 500 ppm fragment ion tolerance. Identified proteins were filtered to using DTASelect and utilizing a target-decoy database search strategy to control the false discovery rate at 1%, at the spectrum level. A minimum of 1 peptide per protein and 1 tryptic end per peptide (no tryptic ends in case of Proteinase K treatment) were required and precursor delta mass cut-off was fixed at 10 ppm. Localization scores were assigned to identified sites of phosphorylation using A-Score. The mass spectrometry proteomics data have been deposited to the ProteomeXchange Consortium ((Deutsch et al, 2020) via the PRIDE (Perez-Riverol et al, 2019; Perez-Riverol et al, 2016) partner repository with the dataset identifier PXD030594 and 10.6019/PXD030594.

Co-immunoprecipitation: HEK293T cells were co-transfected with IRE1 and FMRP plasmids for 24 hours followed by TG (600 nM) or TM (1 μg/ml) for 2 hours. Equal amounts of protein lysates were precipitated with specific antibodies (anti-IRE1 1:250 and anti-FMRP 1:250) at 4° C. overnight on a rocker. Protein magnetic beads were added to each sample and incubated at 4° C. for overnight. Immunoprecipitates were then analyzed by Western Blot.

Kinase assay: HEK293T cells were transfected with either with WT- or KD-IRE1 plasmids for 24 hours, followed by TG (600 nM) for 2 hours for IRE1 activation. Equal amounts of protein lysates were then precipitated with specific IRE1 antibody coated magnetic beads at 4° C. overnight on a rocker. Immunoprecipitates were incubated at 30° C. for 45 min in kinase assay buffer (SignalChem K01-09) with specific ATP analogue (ATP-γ-S, 100 M) and purified hFMRP protein. p-Nitrobenzyl mesylate (PNBM) and 0.5 M ETDA solution was added to reaction after 45 minutes and incubated for additional 2 hours at RT to alkylate the kinase substrate. Samples were boiled with SDS-PAGE loading dye at 95° C. for 5 min to release the proteins from magnetic beads. Beads were separated using magnetic rack and supernatants were analyzed by Western Blot.

Kinase assay for phospho-proteomics: Recombinant active IRE1 (500 ng) and FMRP (500 ng) proteins were incubated in kinase buffer at 30° C. for 45 minutes with ATP-γ-S (100 μM). Samples were then incubated at 24° C. for 1 hour with PNBM (2.5 mM). Samples were boiled in SDS loading buffer at 95° C. and loaded to SDS-polyacrylamide gel (SDS-PAGE) gels. Anti-Thiophosphate ester antibody was used to detect alkylated kinase substrate.

Polysome fractionation: Polysome fractionation protocol was adapted from Stastna et al (Stastna et al, 2018). Briefly, Fmr1^{+/+} and Fmr1^{-/-} BMDM were treated with PA (500 μM) for 6 hours followed by cycloheximide (100 μg/mL) for 10 min prior to lysis with buffer (100 mM KCl, 20 mM Tris pH 7.5, 5 mM MgCl₂, 0.4% NP-40, 100 μg/mL cycloheximide, 0.1 U RNase inhibitor and protease inhibitor cocktail). Clear lysates were loaded to 10-50% sucrose gradient (in Beckman Coulter Thinwall, Ultra-Clear tubes, 344059) and centrifuged (in Beckman LE-80K) for 120 min at 40,000× rpm at 4° C. in a swinging bucket rotor (Beckman SW41) with no-brake. Each gradient was collected as 17 fractions in microcentrifuge tubes with continuous monitoring of absorbance at 254 nm (Biologic LP (pump), Biorad 731-8300; BioFrac, Biorad 741-0002) and frozen immediately at −80° C. for further analysis.

Cholesterol efflux assay: Efflux assay were performed according to manufacturer's instructions (ab196985). Briefly, macrophages were pre-loaded with fluorescently labeled cholesterol for 16 hours in RPMI media including ACAT inhibitor (5 mg/ml), followed by incubation in efflux medium including cholesterol acceptors apolipoprotein A1 (APOA1; 25 μg/ml) or high-density lipoprotein (HDL; 50 μg/ml) for 6 hours. % Efflux was calculated as cholesterol signal in medium/cholesterol signal in medium and cell.

In vitro foam cell formation assay: BMDM were incubated with RPMI containing dil-labeled ac-LDL (25 μg/ml), 10% lipoprotein-deficient serum and 20% L-Glutamine for 24 hours. After cholesterol loading, cells were rinsed with PBS and collected in 2% BSA in PBS. Flow cytometry was performed on a BD Fortessa using FACSDiva software with single stain compensation controls acquired on the same day.

In vivo foam cell formation assay: Fmr1^{+/+} and Fmr1^{-/-} mice were injected with a gain-of-function mutant (D377Y) of proprotein convertase subtilisin kexin 9 (PCSK9)-encoding adeno-associated virus (AAV_mPCSK9) and fed with 16 weeks of WD to induced hypercholesterolemia. Apoe^{-/-} mice were fed with 12 weeks of WD with 4 weeks of AMG-18 (30 mg/kg, once a day) injection during the last 4 weeks of WD. The peritoneal macrophages collected and assessed for lipid accumulation by Oil-Red O and Hematoxylin staining.

Preparation of radiolabel cholesterol: ag-LDL (50 μg/mL, made in house with endotoxin free LDL isolated from human plasma) and [³H]-cholesterol (5 μCi/mL) were incubated for 1 hour at 37° C. in a sterile endotoxin free bottle.

RCT assay: Fmr1^{+/+} and Fmr1^{-/-} BMDM were incubated with radiolabeled ag-LDL for 30 hours followed by warm HBSS wash and equilibration in 2 mg/mL fatty-acid free BSA overnight. Cells were washed twice in ice cold HBSS and incubated with EDTA (5 mM) for 20 min at 4° C. and spun down at 200×g for 5 min. Cells were resuspended in ice cold DMEM and injected into C57BL6N mice subcutaneously in the scruff of the neck. Blood was collected at 24 hours via the saphenous vein and at 48 hours via cardiac puncture of anesthetized mice. Plasma was used for liquid scintillation counting. At 48 hours livers were removed for scintillation counting. Feces were collected over a 48 hours period, and total feces radioactivity was measured. All [³H]-tracer measurements are expressed relative to the injected amount.

Induction of apoptosis and labeling of Jurkat cells: Jurkat cells were fluorescently labeled with CellTrace CSFE or Violet (2 μM) in PBS for 20 min. Cells were then washed ones with PBS and seeded in conditioned DMEM medium followed by irradiation under a 254 nm UV lamp for 5 min. Cells were incubated under normal cell culture conditions for 3-4 hours. Apoptosis was confirmed by Anexin V⁺ staining (minimum 85% Annexin V⁺ cells). The apoptotic cells (ACs) were centrifuged at 500×g for 5 min and resuspended in conditioned DMEM for experiments.

In vitro efferocytosis: Bone marrow-derived macrophages were plated in 6-well dishes at a density of 0.5×10⁶ cells per well. CSFE-labeled ACs were incubated with the macrophages for 2-4 min at a 5:1 AC: macrophage ratio followed by washing three times with PBS. Some groups of macrophages were then incubated for another 2 hours in normal cell culture media, followed by the addition of Violet-labeled ACs. After 2 hours, macrophages were washed three times with PBS to remove unbound ACs, and then the macrophages were fixed with 4% formaldehyde for 20 min, rinsed three times with PBS, blocked by TruStain FcX™ (anti-mouse CD16/32) for 10 min and then stained with PE-F4/80 o/n. The percentage of PE-F4/80⁺ and CFSE⁺ double positive cells to total PE-F4/80⁺ cells were reported as % efferocytosis and PE-F4/80⁺, CFSE⁺ and Violet⁺ triple positive cells to PE-F4/80⁺ and CFSE⁺ double positive cells were reported as % continuous efferocytosis.

In vivo efferocytosis: Fmr1^{-/-} or Fmr1^{-/-} mice were fed with WD for 16 weeks and injected with 1×10⁶ CFSE-labeled ACs and 1.5 hours later subsequently peritoneal lavages were collected and stained for PE-F4/80⁺ resident macrophages. The percentage of PE-F4/80⁺ and CFSE⁺ double positive cells to total PE-F4/80⁺ cells were reported as % efferocytosis. Another group of C57BL/6 were injected with AMG-18 (30 mg/kg) or vehicle (DMSO). After 8 hours both groups were interperitoneally injected with CFSE-labeled ACs and 1.5 hours later peritoneal lavages were collected and cultured for 30 minutes to allow cells to attach. Macrophages were washed three times with PBS to remove unbound ACs, and then the macrophages were fixed with 4% formaldehyde for 20 min, rinsed three times with PBS, blocked by TruStain FcX™ (anti-mouse CD16/32) for 10 minutes and then stained with PE-F4/80 o/n. The percentage of F4/80⁺ and CFSE⁺ double positive cells to total F4/80⁺ cells were reported as % efferocytosis.

En face Oil-Red O staining: Aortas opened longitudinally were rinsed with 60% isopropanol for 1 min, stained with Oil-Red O solution for 20 min and then distained in 60% isopropanol for 1 min and rinsed in PBS. The lesion area was quantified as percent of Oil-Red O staining area in total aorta area.

Immunohistochemistry: 7 μm thick aortic root cryosections (from OCT embedded heart tissue) were stained with antibodies for: anti-MOMA-2 (1:500) and anti-α-SMA (1:500) and images were captured with fluorescent microscope. Cryosections were stained with Masson's Trichrome, TUNEL, Hematoxylin and Eosin (H&E) according to manufacturer's instructions. Cryosections were stained with H&E for morphometric lesion analysis. The total lesion area and necrotic area were quantified as previously described from 4 sequential sections (60 μM apart, beginning at the base of the aortic root). Foam cell area was calculated from Oil-Red O stained 4 sequential sections (60 μM apart, beginning at the base of the aortic root) and collagen content from Masson's Trichrome stained sections using ImageJ.

The fluorescent immunostainings were carried out on cryosections that were fixed in cold acetone for 10 minutes, blocked in goat serum/BSA/PBS as previously described. All stained sections were mounted with fluoroshield mounting reagent with DAPI. Fluorescent signal calculations: (a) TUNEL staining: the sections were double stained with MOMA-2 to mark the macrophage-enriched area. The Mean Fluorescent Intensity (MFI) corresponding to primary antibody signal was calculated from the MOMA-2 positive area. The background fluorescence of the non-stained area inside the lesion was subtracted from the total MFI corresponding to each signal (b) α-SMA staining: α-SMA positive area was calculated from the plaque area. The background fluorescence of the non-stained area inside the lesion was subtracted from the total MFI corresponding to each signal. Data were quantified as total MFI signal compared with baseline.

Apoptosis detection by flow cytometry: Apoptosis was induced after treatments by PA (500 μM) treatment for 12 hours. Fc receptors were blocked by pre-incubating cells with 0.25 μg of TruStain FcX™ PLUS (anti-mouse CD16/32) Antibody per 10⁶ cells for 5-10 minutes on ice. Cells were then incubated with PI on ice for 20 minutes in the dark followed by 2× with 2 ml of cell staining buffer. Cells were resuspended in 500 μl of cell staining buffer and analyzed on a BD Fortessa using FACSDiva software with single stain compensation controls acquired on the same day. Data were analyzed using FlowJo analysis software (FlowJo, LLC).

Flow cytometric analysis of peripheral blood: 100 μl of blood was collected in EDTA-coated tubes and red blood cells were removed by incubation (3×) in Ammonium-Chloride-Potassium (ACK, Thermo Fisher A1049201) solution for 5 min at room temperature. Peripheral blood mononuclear cells were then resuspended in FACS buffers (2% BSA in PBS) and incubated for 20 min on ice with the following antibodies: anti-CD45-Pac. Blue (clone 30-F11), CD3e-PE (Clone 145-2C11), CD11b-APC (Clone M1/70), CD19-BV650 (Clone 6D5), Ly6C-PE/Dazzle (Clone HK1.4) and Ly6G-PerCP Cy5.5 (1A8) in 1:100 dilution ratio. Stained samples were washed once and resuspended in FACS buffer containing DAPI (4 μg/ml). Flow cytometry was performed on a BD Fortessa using FACSDiva software with single stain compensation controls acquired on the same day. Data were analyzed using FlowJo analysis software (FlowJo, LLC). All antibodies were purchased from Biolegend (San Diego, CA) and used at the manufacturer's recommended concentrations.

Plasma lipids and lipoprotein analysis: Plasma was analyzed by FPLC in the Department of Internal Medicine/Lipid Science, Wake Forest University School of Medicine Winston-Salem, NC 27019. The total cholesterol and triglyceride measurement were performed using WAKO Cholesterol E kit according to the manufacturer's instructions.

Statistics: Results are reported as mean±SEM and statistical significance was determined with Unpaired t-test with Welch's or Mann-Whitney correction test by GraphPad Software, LLC. * P≤0.05, ** P≤0.01, *** P≤0.001 was considered as * significant.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

1. A method for reducing the likelihood or inhibiting progression of atherosclerosis, or reducing the severity or treating atherosclerosis, in a subject in need thereof, comprising administering to the subject a composition comprising a therapeutically effective amount of an inhibitor of inositol-requiring enzyme-1 (IRE1), an inhibitor of fragile X mental retardation protein (FMRP), or both.

2. (canceled)

3. The method of claim 1, wherein the inhibitor of IRE1 is a small molecule that inhibits kinase activity of the IRE1, wherein in the presence of the IRE1 and the inhibitor of IRE1, phosphorylation of FMRP is reduced compared to that in the presence of the IRE1 without the inhibitor of IRE1.

4. The method of claim 3, wherein the inhibitor of IRE1 comprises ORIN1001, MCK8866, AMG-18, G-9807, G-1749, STF-083010, or 4μ8C.

5. The method of claim 3, wherein the inhibitor of IRE1 is a kinase-specific inhibitor of IRE1, comprising AMG-18.

6. The method of claim 1, wherein the inhibitor of IRE1 inhibits level of IRE1 by suppressing expression of ERN1 gene, wherein the inhibitor is a small interfering RNA (siRNA), a microRNA (miRNA) or an antisense oligo, wherein the siRNA, the miRNA, or the antisense oligo targets the mRNA of ERN1 and suppresses expression of at least kinase domain of ERN1.

7. The method of claim 1, wherein the inhibitor of FMRP inhibits level of FMRP by suppressing expression of FMRP gene, wherein the inhibitor is an siRNA, a miRNA, or an antisense oligo, wherein the siRNA, the miRNA, or the antisense oligo targets FMR1 and suppresses expression of FMR1.

8. (canceled)

9. The method of claim 1, wherein the subject has obesity.

10. The method of claim 1, wherein the subject has hyperlipidemia or hypercholestorelimia.

11. The method of claim 1, wherein the composition is administered once, twice, or three times per day to the subject until one or more improvements is observed, said improvements comprising reduced atherosclerotic lesion, reduced foam cell area in the atherosclerotic lesion, and/or reduced necrotic core area of the atherosclerotic lesion, in a blood vessel of the subject.

12. A method for screening for an atheroprotective agent that inhibits kinase activity of IRE1, inhibits binding between IRE1 and FMRP, or reduces phosphorylation of FMRP, comprising:
contacting a molecule of interest with an atherosclerotic lesion cell type, said cell type expressing IRE1 and FMRP, and
measuring an amount of phosphorylation of the FMRP in the cell type,
wherein a decrease in the amount of phosphorylation of the FMRP relative to an amount before the contact with the molecule of interest is indicative that the molecule of interest is an atheroprotective agent, and
wherein the atherosclerotic lesion cell type comprises macrophage, endothelial cell, or vascular smooth muscle cell.

13. The method of claim 12, further comprising stressing the atherosclerotic lesion cell type by contacting the atherosclerotic lesion cell type with a stressor, said stressor comprising a saturated fatty acid, an oxidized low density lipoprotein (oxLDL), thapsigargin, or tunicamycin.

14. The method of claim 12, wherein the atherosclerotic lesion cell type is macrophage.

15. A method for enhancing macrophage's activity in engulfing apoptotic cells for clearance, exporting cholesterol to lipid-poor apolipoprotein, and/or internalizing low density lipoprotein (LDL), reducing the likelihood of foam cell formation from the macrophage, and/or reducing atherosclerotic lesion or necrotic core area of the atherosclerotic lesion in a blood vessel, said atherosclerotic lesion comprising the macrophage, the method comprising contacting the macrophage with an inhibitor of IRE1, an inhibitor of FMRP, or both; or transfecting or expressing a vector encoding the inhibitor of IRE1, the inhibitor of FMRP, or both in the macrophage.

16. The method of claim 15, wherein the inhibitor of IRE1 is a small molecule that inhibits kinase activity of the IRE1, wherein in the presence of the IRE1 and the inhibitor of IRE1, phosphorylation of FMRP is reduced compared to that in the presence of the IRE1 without the inhibitor of IRE1.

17. The method of claim 16, wherein the inhibitor of IRE1 comprises ORIN1001, MCK8866, AMG-18, G-9807, G-1749, STF-083010, or 4μ8C.

18. The method of claim 15, wherein the inhibitor of IRE1 inhibits level of IRE1 by suppressing expression of ERN1 gene, wherein the inhibitor is a small interfering RNA (siRNA), a microRNA (miRNA) or an antisense oligo, wherein the siRNA, the miRNA, or the antisense oligo targets the mRNA of ERN1 and suppresses expression of at least kinase domain of ERN1.

19. The method of claim 15, wherein the inhibitor of FMRP inhibits level of FMRP by suppressing expression of FMRP gene, wherein the inhibitor is an siRNA, a miRNA, or an antisense oligo, wherein the siRNA, the miRNA, or the antisense oligo targets FMR1 and suppresses expression of FMR1.

20. The method of claim 15, wherein the subject has obesity.

21. The method of claim 15, wherein the subject has hyperlipidemia or hypercholestorelimia.

22. The method of claim 15, wherein the inhibitor of FMRP is a phosphatase which reduces pFMRP/FMRP ratio in the macrophage.


SEQUENCE LISTING (47 sequences)

SEQ ID NO: 1 - protein, 663 aa, Homo sapiens
  MOD_RES 362: note = PHOSPHORYLATION
  MOD_RES 494: note = PHOSPHORYLATION
  MOD_RES 497: note = PHOSPHORYLATION
  MOD_RES 500: note = PHOSPHORYLATION
  MOD_RES 502: note = PHOSPHORYLATION
  MOD_RES 504: note = PHOSPHORYLATION
  MOD_RES 592: note = PHOSPHORYLATION
  MOD_RES 594: note = PHOSPHORYLATION
MEELVVEVRGSNGAFYKAFVKDVHEDSITVAFENNWQPDRQIPFHDVRFPPPVGYNKDINESDEVEVYSRANEKEPCCWWLAKVRMIKGEFYVIEYAACDATYNEIVTIERLRSVNPNKPATKDTFHKIKLDVPEDLRQMCAKEAAHKDFKKAVGAFSVTYDPENYQLVILSINEVTSKRAHMLIDMHFRSLRTKLSLIMRNEEASKQLESSRQLASRFHEQFIVREDLMGLAIGTHGANIQQARKVPGVTAIDLDEDTCTFHIYGEDQDAVKKARSFLEFAEDVIQVPRNLVGKVIGKNGKLIQEIVDKSGVVRVRIEAENEKNVPQEEEIMPPNSLPSNNSRVGPNAPEEKKHLDIKENSTHFSQPNSTKVQRVLVASSVVAGESQKPELKAWQGMVPFVFVGTKDSIANATVLLDYHLNYLKEVDQLRLERLQIDEQLRQIGASSRPPPNRTDKEKSYVTDDGQGMGRGSRPYRNRGHGRRGPGYTSGTNSEASNASETESDHRDELSDWSLAPTEEERESFLRRGDGRRRGGGGRGQGGRGRGGGFKGNDDHSRTDNRPRNPREAKGRTTDGSLQIRVDCNNERSVHTKTLQNTSSEGSRLRTGKDRNQKKEKPDSVDGQQPLVNGVPTRTRPLEQKLISEEDLAANDILDYKDDDDKV

SEQ ID NO: 2 - protein, 18 aa, Homo sapiens
  MOD_RES 8: note = PHOSPHORYLATION
HLDIKENSTHFSQPNSTK

SEQ ID NO: 3 - protein, 19 aa, Homo sapiens
  MOD_RES 9: note = PHOSPHORYLATION
KHLDIKENSTHFSQPNSTK

SEQ ID NO: 4 - protein, 19 aa, Homo sapiens
  MOD_RES 9: note = PHOSPHORYLATION
KHLDIKENSTHFSQPNSTK

SEQ ID NO: 5 - protein, 16 aa, Homo sapiens
  MOD_RES 11: note = PHOSPHORYLATION
RGPGYTSGTNSEASNA

SEQ ID NO: 6 - protein, 40 aa, Homo sapiens
  MOD_RES 14: note = PHOSPHORYLATION
RGPGYTSGTNSEASNASETESDHRDELSDWSLAPTEEERE

SEQ ID NO: 7 - protein, 14 aa, Homo sapiens
  MOD_RES 1: note = PHOSPHORYLATION
SETESDHRDELSDW

SEQ ID NO: 8 - protein, 16 aa, Homo sapiens
  MOD_RES 3: note = PHOSPHORYLATION
NASETESDHRDELSDW

SEQ ID NO: 9 - protein, 16 aa, Homo sapiens
  MOD_RES 3: note = PHOSPHORYLATION
NASETESDHRDELSDW

SEQ ID NO: 10 - protein, 16 aa, Homo sapiens
  MOD_RES 3: note = PHOSPHORYLATION
NASETESDHRDELSDW

SEQ ID NO: 11 - protein, 40 aa, Homo sapiens; identical to SEQ ID NO: 6
  MOD_RES 19: note = PHOSPHORYLATION

SEQ ID NO: 12 - protein, 14 aa, Homo sapiens
  MOD_RES 5: note = PHOSPHORYLATION
SETESDHRDELSDW

SEQ ID NO: 13 - protein, 16 aa, Homo sapiens
  MOD_RES 4: note = PHOSPHORYLATION
SVHTKTLQNTSSEGSR

SEQ ID NO: 14 - protein, 16 aa, Homo sapiens
  MOD_RES 4: note = PHOSPHORYLATION
SVHTKTLQNTSSEGSR

SEQ ID NO: 15 - protein, 16 aa, Homo sapiens
  MOD_RES 4: note = PHOSPHORYLATION
SVHTKTLQNTSSEGSR

SEQ ID NO: 16 - protein, 16 aa, Homo sapiens
  MOD_RES 6: note = PHOSPHORYLATION
SVHTKTLQNTSSEGSR

SEQ ID NO: 17 - RNA, 21 nt, synthetic construct
  source 1..21: mol_type = other RNA
  modified_base 20: mod_base = OTHER; note = thymine
  modified_base 21: mod_base = OTHER; note = thymine
gcatgtgatgctacgtatatt

SEQ ID NO: 18 - RNA, 21 nt, synthetic construct
  source 1..21: mol_type = other RNA
  modified_base 20: mod_base = OTHER; note = thymine
tatacgtagcatcacatgctg

SEQ ID NO: 19 - RNA, 21 nt, synthetic construct
  source 1..21: mol_type = other RNA
  modified_base 20: mod_base = OTHER; note = thymine
  modified_base 21: mod_base = OTHER; note = thymine
ggtgccagaagatttacgatt

SEQ ID NO: 20 - RNA, 21 nt, synthetic construct
  source 1..21: mol_type = other RNA
  modified_base 20: mod_base = OTHER; note = thymine
tcgtaaatcttctggcacctc

SEQ ID NO: 21 - DNA, 16 bp, synthetic construct
  source 1..16: mol_type = other DNA
ggaataagaattacgg

SEQ ID NO: 22 - DNA, 21 bp, synthetic construct
  source 1..21: mol_type = other DNA
ccgaacagataatcgtccacg

SEQ ID NO: 23 - DNA, 21 bp, synthetic construct
  source 1..21: mol_type = other DNA
acgctgtctggcttttccttc

SEQ ID NO: 24 - DNA, 21 bp, synthetic construct
  source 1..21: mol_type = other DNA
aaaaccgcagacatccttcag

SEQ ID NO: 25 - DNA, 21 bp, synthetic construct
  source 1..21: mol_type = other DNA
cataccgaaactcgttcaccc

SEQ ID NO: 26 - DNA, 20 bp, synthetic construct
  source 1..20: mol_type = other DNA
ggtcctgacacatctgcgaa

SEQ ID NO: 27 - DNA, 20 bp, synthetic construct
  source 1..20: mol_type = other DNA
caggaccttcttggcttcgt

SEQ ID NO: 28 - DNA, 20 bp, synthetic construct
  source 1..20: mol_type = other DNA
cagggcctttaccagggaga

SEQ ID NO: 29 - DNA, 21 bp, synthetic construct
  source 1..21: mol_type = other DNA
tgtgtgctggatgtgatcttc

SEQ ID NO: 30 - DNA, 21 bp, synthetic construct
  source 1..21: mol_type = other DNA
gcctacacctggagagatagc

SEQ ID NO: 31 - DNA, 19 bp, synthetic construct
  source 1..19: mol_type = other DNA
ggcaacttacgagcaggct

SEQ ID NO: 32 - DNA, 20 bp, synthetic construct
  source 1..20: mol_type = other DNA
gtgctctcccttgattctgc

SEQ ID NO: 33 - DNA, 20 bp, synthetic construct
  source 1..20: mol_type = other DNA
ctgcaccaataacagctcca

SEQ ID NO: 34 - DNA, 21 bp, synthetic construct
  source 1..21: mol_type = other DNA
tggtgggaaactacacttgcg

SEQ ID NO: 35 - DNA, 21 bp, synthetic construct
  source 1..21: mol_type = other DNA
cgtgcggtttttcagctctat

SEQ ID NO: 36 - DNA, 21 bp, synthetic construct
  source 1..21: mol_type = other DNA
gcagaccctgccatctatttc

SEQ ID NO: 37 - DNA, 23 bp, synthetic construct
  source 1..23: mol_type = other DNA
tcggacttatgtttggattcgac

SEQ ID NO: 38 - DNA, 19 bp, synthetic construct
  source 1..19: mol_type = other DNA
atgcaggccatcaagtgtg

SEQ ID NO: 39 - DNA, 20 bp, synthetic construct
  source 1..20: mol_type = other DNA
taggagaggggacgcaatct

SEQ ID NO: 40 - DNA, 25 bp, synthetic construct
  source 1..25: mol_type = other DNA
caaccaacaagtgatattctccatg

SEQ ID NO: 41 - DNA, 21 bp, synthetic construct
  source 1..21: mol_type = other DNA
gatccacactctccagctgca

SEQ ID NO: 42 - DNA, 20 bp, synthetic construct
  source 1..20: mol_type = other DNA
attcaacggcacagtcaagg

SEQ ID NO: 43 - DNA, 20 bp, synthetic construct
  source 1..20: mol_type = other DNA
tggatgcagggatgatgttc

SEQ ID NO: 44 - DNA, 39 bp, synthetic construct
  source 1..39: mol_type = other DNA
gcatcaaatgctgctgaagcagaagctgaccacagagac

SEQ ID NO: 45 - DNA, 39 bp, synthetic construct
  source 1..39: mol_type = other DNA
gtctctgtggtcagcttctgcttcagcagcatttgatgc

SEQ ID NO: 46 - DNA, 39 bp, synthetic construct; identical to SEQ ID NO: 44
  source 1..39: mol_type = other DNA

SEQ ID NO: 47 - DNA, 39 bp, synthetic construct; identical to SEQ ID NO: 45
  source 1..39: mol_type = other DNA